(19) United States
(12) Patent Application Publication
(10) Pub. No.: US 2006/0141444 A1
(43) Pub. Date: Jun. 29, 2006

(54) Adherent entities and uses therefor

(75) Inventors: Christine Letitia Knox, Queensland (AU); Peter Timms, Queensland (AU)

Correspondence Address: AKIN GUMP STRAUSS HAUER & FELD L.L.P., ONE COMMERCE SQUARE, 2005 MARKET STREET, SUITE 2200, PHILADELPHIA, PA, 19103, US

(73) Assignee: Queensland University Of Technology

(21) Appl. No.: 10/715,926

(22) Filed: Nov. 18, 2003

Related U.S. Application Data
(63) Continuation of application No. PCT/AU02/00611, filed on May 17, 2002.

(30) Foreign Application Priority Data
May 18, 2001 (AU) PR 5124

(51) Int. Cl. C12Q 1/00

(52) U.S. Cl. 435/4

(57) ABSTRACT

The present invention is directed to reproductive function-modulating organisms especially of the genera Ureaplasma and Mycoplasma, which adhere more strongly to spermatozoa than other organisms and which correlate with the presence or risk of a condition selected from male infertility, an adverse pregnancy outcome or an adverse assisted reproductive technology outcome. More particularly, the invention is directed to the detection of these reproductive function-modulating organisms (also referred to herein as adherovars), and to various therapeutic and prophylactic strategies for enhancing or otherwise improving inter alia fertility and especially male fertility.

### CROSS-REFERENCE TO RELATED APPLICATIONS

This application is a continuation of International Application No. PCT/AU02/00611, filed May 17, 2002, which was published in the English language on Nov. 28, 2002, under International Publication No. WO 02/095410 A1, the disclosure of which is incorporated herein by reference.

### BACKGROUND OF THE INVENTION

This invention relates generally to pathogenic agents associated with genital tract infections. More particularly, the present invention relates to reproductive function-modulating organisms of the class Mollicutes, especially of the order Mycoplasmatales, more especially of the family Mycoplasmataceae, even more especially of the genera Mycoplasma and Ureaplasma, particularly strains or serotypes of the species Ureaplasma parvum, U. urealyticum and Mycoplasma hominis, which adhere more strongly to spermatozoa than other members of the aforementioned class, order, family genus, strain or serotype and which correlate with the presence or risk of a condition selected from male infertility, an adverse pregnancy outcome or an adverse assisted reproductive technology outcome. Even more particularly, the invention relates to the detection of these reproductive function-modulating organisms (also referred to herein as adherovars), and to various therapeutic and prophylactic strategies for enhancing or otherwise improving inter alia fertility and especially male fertility. The invention also extends to an adhesin present on the surface of these adherovars, to methods for its preparation and to adhesin-encoding polynucleotides. The present invention also extends to the development of methods for screening of agents useful inter alia for modulating an adhesin-encoding gene or for modulating the level and/or functional activity of an expression product of that gene. The invention also concerns the use of adhesin-containing compositions in the production of antigen-binding molecules that are immuno-interactive with the adhesin. The invention further relates to the use of the adhesin, or a biologically-active fragment thereof, or a variant or derivative of these, and to the use of above modulatory agents in the preparation of compositions for the treatment and/or prophylaxis of infections caused by the aforesaid adherovars, for the treatment of spermatozoa and for improving spermatozoal fertilization of oocytes.

Bibliographic details of various publications referred to in this specification are collected at the end of the description.

The ureaplasmas, the newly designated U. parvum (previously U. urealyticum serovars 1, 3, 6, and 14) and U. urealyticum (previously serovars 2, 4; 5, 7-13) are the microorganisms most frequently isolated from placental tissue with histological evidence of chorioamnionitis. This upper genital tract infection, frequently clinically asymptomatic, is significantly associated with adverse pregnancy sequelae including preterm delivery, premature onset of labor, prolonged rupture of membranes and neonatal morbidity and mortality (Cassell, et al., 1993).

Ascending infection of the placenta occurs rapidly upon rupture of membranes but mid-trimester, persistent ureaplasma amnionitis has also been reported in the absence of premature labor or rupture of placental membranes (Cassell, et al., 1986) (Cassell, et al., 1983). In a previous study, the present inventors subtyped ureaplasma isolates from infected placental tissue and from the female lower genital tract and detected different ureaplasma subtypes in these sites (Knox and Timms, 1998). They have also shown that the male urogenital tract and the female lower genital tract may be separately colonized with different ureaplasma subtypes. These findings are consistent with an exogenous reservoir, the male urogenital tract, as the source of upper genital tract infection (of the placenta and the foetus) in pregnant women.

Several studies have investigated the effect of ureaplasma positive semen samples on assisted reproductive technology (ART) outcomes, however, the results of these studies have been inconsistent. Some studies but not all found (i) reduced pregnancy rates per embryo transfer (Montagut, et al., 1991; Shalika, et al., 1996) (ii) reduced blastocyst culture rates (Riedel, et al., 1986) and (iii) an increased miscarriage rate (Kanakas, et al., 1999) when the male semen was infected or colonized with ureaplasmas.

In work leading up to the present invention, the inventors studied the effect of ureaplasma positive washed semen on: fertilization by in vitro fertilization (IVF) or by intracytoplasmic injection (ICSI); and on ART clinical outcomes, the viable pregnancy rate and the miscarriage rate. The effect of different ureaplasma subtypes on these outcomes was also examined. This study (results for 412 embryo transfers) demonstrated a reduced viable pregnancy rate per embryo transfer (5%) in ICSI couples with ureaplasma positive washed semen, compared to ICSI couples with ureaplasma positive semen, negative washed semen (23%), and ICSI couples with ureaplasma negative semen and washed semen (14%). This study also showed an increase in the miscarriage rate (miscarriages/no clinical pregnancies) for ureaplasma washed semen positive couples after ICSI (66%) and IVF (50%) insemination procedures compared to couples with ureaplasma positive semen, negative washed semen (20% and 25%, respectively), and couples with ureaplasma negative semen and washed semen (26% and 35.5%, respectively). By contrast a comparison of endocervical ureaplasma positive couples and endocervical ureaplasma negative couples showed no differences in the viable pregnancy rate per embryo transfer (15% and 13% respectively) or in the miscarriage rate (28% and 37.5%, respectively).

Unexpectedly, this investigation also revealed that certain subtypes of strains or serotypes of Ureaplasma and Mycoplasma adhere more strongly to spermatozoa than other subtypes of this genus and, consequently, remain adherent to spermatozoa after washing. Taken together, these results indicate that these more strongly adherent members of Ureaplasma and Mycoplasma are more likely to impact adversely on ART pregnancy outcomes, and strongly support the hypothesis that ureaplasma adherence to spermatozoa is a mechanism of pathogenicity facilitating infection of the embryo at conception. The inventors have also determined that other organisms of the class Mollicutes, especially of the order Mycoplasmatales and more especially of the family Mycoplasmataceae, remain adherent to spermatozoa after washing and that such organisms would be expected, therefore, to also impact adversely on ART pregnancy outcomes. Strongly adherent organisms of this nature, which correlate with higher rates of infertility or lower ART pregnancy outcomes, are also referred to collectively herein as adherovars. The aforementioned discoveries have been reduced to practice inter alia in methods for detecting strongly spermatozoal adherent organisms that modulate reproductive function, in methods of diagnosis of conditions associated with these organisms and in various therapeutic and prophylactic strategies for enhancing or otherwise improving fertility, and especially male fertility, as described hereinafter.

### BRIEF SUMMARY OF THE INVENTION

Accordingly, in one aspect of the present invention, there is provided a method for detecting the presence of a reproductive function-modulating organism, comprising detecting an organism which is capable of adhering to sperm and which correlates with the presence or risk of a condition selected from male infertility, an adverse pregnancy outcome or an adverse assisted reproductive technology (ART) outcome.

The correlation with the presence or risk of the condition is preferably made if the organism is capable of remaining adherent to sperm after washing a sample comprising organism-positive sperm to permit removal or separation of non-sperm substances from the sample. In a preferred embodiment, the correlation with the presence or risk of the condition is made if the washed sperm sample comprises at least 0.01% of the total number of organisms present in said sperm sample before washing.

The washing is preferably carried out using wash conditions that protect one or more viability properties of the sperm. In one embodiment, the wash conditions are characterised in that they permit the continuation of adherence of Ureaplasma parvum serotype 6 to said sperm but do not permit the continuation of adherence of U. parvum serotype 1 to said sperm. In a preferred embodiment, the washing is carried out using a sperm swim-up technique. In another preferred embodiment, the washing is carried out using gradient centrifugation.

The organism is suitably selected from the class Mollicutes, especially from the order Mycoplasmatales, more especially from the family Mycoplasmataceae, and even more especially from the genus Mycoplasma and even more especially from the genus Ureaplasma. In a preferred embodiment, the organism is a subtype of a strain or serotype of Ureaplasma parvum or Ureaplasma urealyticum. In an especially preferred embodiment of this type, the organism is a subtype of Ureaplasma parvum serotype 6. In another preferred embodiment, the organism is a subtype of a strain or serotype of Mycoplasma hominis.

In another aspect, the invention contemplates a method for detecting the presence of a reproductive function-modulating organism, comprising detecting an organism which is capable of adhering to sperm and which correlates with the presence or risk of male infertility.

In yet another aspect, the invention encompasses a method for detecting the presence of a reproductive function-modulating organism, comprising detecting an organism which is capable of adhering to sperm and which correlates with the presence or risk of an adverse pregnancy outcome.

In still another aspect, the invention extends to a method for detecting the presence of a reproductive function-modulating organism, comprising detecting an organism which is capable of adhering to sperm and which correlates with the presence or risk of an adverse assisted reproductive technology (ART) outcome.

In a further aspect, the invention provides a method for detecting an organism associated with male infertility or with an increased risk of male infertility, comprising detecting an organism which is capable of adhering to sperm and which correlates with the presence or risk of male infertility.

In yet a further aspect, the invention contemplates a method for detecting an organism associated with an adverse pregnancy outcome or with an increased risk of an adverse pregnancy outcome, comprising detecting an organism which is capable of adhering to sperm and which correlates with the presence or risk of said adverse pregnancy outcome.

In still a further aspect, the invention envisions a method for detecting an organism associated with an adverse assisted reproductive technology (ART) outcome or with an increased risk of an adverse ART outcome, comprising detecting an organism which is capable of adhering to sperm and which correlates with the presence or risk of said adverse ART outcome.

In a further aspect, the invention features a method for detecting the presence or diagnosing the risk of infertility in a male patient, comprising detecting in a sperm sample obtained from said patient an organism which is capable of adhering to sperm and which correlates with the presence or risk of male infertility.

In still yet a further aspect, the invention contemplates a method for detecting the presence or diagnosing the risk of an adverse pregnancy outcome in a patient, comprising detecting an organism in a sperm sample obtained from a sperm donor of the patient, wherein the organism is capable of adhering to sperm and correlates with the presence or risk of an adverse pregnancy outcome.

In yet another aspect, the invention encompasses a method for detecting the presence or diagnosing the risk of an adverse assisted reproductive technology (ART) outcome, comprising detecting the presence of an organism in a sperm sample used for said ART, wherein the organism is capable of adhering to sperm and correlates with the presence or risk of an adverse ART outcome. In a preferred embodiment, the ART is selected from artificial insemination (AI), in vitro fertilization (IVF) and intracytoplasmic sperm injection (ICSI).

In yet another aspect, the invention features a method for detecting the presence or diagnosing a condition selected from male infertility, an adverse pregnancy outcome or an adverse assisted reproductive technology (ART) outcome in a patient, comprising detecting an organism in a biological sample obtained from the patient, wherein said organism is capable of adhering to sperm and correlates with the presence or risk of said condition.

Suitably, the biological sample comprises a biological fluid selected from whole blood, serum, plasma, saliva, urine, sweat, ascitic fluid, peritoneal fluid, synovial fluid, and cerebrospinal fluid, amniotic fluid, seminiferous tubule fluid (e.g., in vasectomized men), semen, vaginal secretions, endocervical secretions, respiratory secretions, endometrial lining, fallopian tube washings, follicular fluid. Alternatively, the biological sample comprises a tissue biopsy including, but not restricted to, spermatozoa, endocervical cells, placental tissue, endometrial biopsies, chorioamnion, seminiferous tubules, ovarian tissue, oocytes, embryos, single cells biopsied from embryos and fallopian tube tissue.

In another aspect, the invention envisions a method for diagnosing a higher risk of infertility in a male patient, comprising detecting in a biological sample obtained from said patient an organism which is capable of adhering to sperm and which correlates with a higher risk of male infertility.

In yet another aspect, the invention contemplates a method for diagnosis of a higher risk of an adverse pregnancy outcome in a patient, comprising detecting an ureaplasma in a biological sample obtained from a sperm donor of said patient, wherein said organism is capable of adhering to sperm and correlates with a higher risk of an adverse pregnancy outcome.

In yet another aspect, the invention encompasses a method for diagnosis of a higher risk of an adverse assisted reproductive technology (ART) outcome using sperm of a patient, comprising detecting an organism in a biological sample obtained from said patient, wherein said organism is capable of adhering to sperm and correlates with a higher risk of an adverse ART outcome.

The present inventors consider that the enhanced adherence to, or affinity for, spermatozoa displayed by the above-mentioned organisms is mediated by one or more adhesins present on the surface of those organisms. Accordingly, in another aspect, the invention contemplates an isolated adhesin, or a biologically active fragment thereof, or a variant or derivative of these, wherein said adhesin is obtainable from an organism which is capable of adhering to sperm and which correlates with the presence or risk of a condition selected from male infertility, an adverse pregnancy outcome or an adverse assisted reproductive technology (ART) outcome.

In another aspect, the invention encompasses an isolated polynucleotide encoding the adhesin, or the biologically active fragment thereof, or the variant or derivative of these, as broadly described above.

In yet another aspect of the present invention, there is provided a method for detecting an organism associated with male infertility or with an increased risk of male infertility, comprising interrogating a microbial sample for the presence of a gene encoding an adhesin or an expression product of said gene, wherein said adhesin is expressed by an organism which is capable of adhering to sperm and which correlates with the presence or risk of male infertility.

In still yet another aspect, the invention resides in a method for detecting an organism associated with male infertility or with an increased risk of male infertility, comprising interrogating a microbial sample for the presence of a gene encoding an adhesin or an expression product of said gene, wherein said adhesin or variant thereof is expressible by an organism which is capable of adhering to sperm and which correlates with the presence or risk of male infertility.

In a further aspect, the invention encompasses a method for detecting an ureaplasma associated with an adverse pregnancy outcome or with an increased risk of an adverse pregnancy outcome, comprising interrogating a microbial sample for the presence of a gene encoding an adhesin or an expression product of said gene, wherein said adhesin or variant thereof is expressible by an organism which is capable of adhering to sperm and which correlates with the presence or risk said adverse pregnancy outcome.

In another aspect, the invention envisions a method for detecting an ureaplasma associated with an adverse assisted reproductive technology (ART) outcome or with an increased risk of an adverse ART outcome, comprising interrogating a microbial sample for the presence of a gene encoding an adhesin or an expression product of said gene, wherein said adhesin or variant thereof is expressible by an organism which is capable of adhering to sperm and which correlates with the presence or risk said adverse ART outcome.

The present inventors consider that the adhesin as broadly described above, as well as the gene encoding that adhesin, can be used to provide both drug targets and regulators to promote or inhibit the adherence of said adhesin to spermatozoa and to provide diagnostic markers for male infertility, adverse pregnancy outcomes or adverse assisted reproductive technology (ART) outcomes, using, for example, detectable polypeptides and polynucleotides as broadly described above, or using detectable agents which interact specifically with those polypeptides or polynucleotides.

Thus, in another aspect, the invention extends to a method of screening for an agent which modulates the adherence of an organism to spermatozoa, wherein said organism is capable of adhering to sperm and correlates with the presence or risk of a condition selected from male infertility, an adverse pregnancy outcome or an adverse assisted reproductive technology (ART) outcome, said method comprising:
contacting a preparation comprising:

(i) a polypeptide comprising an amino acid sequence corresponding to at least a biologically active fragment of said adhesin, or to a variant or derivative thereof; or

(ii) a polynucleotide comprising at least a portion of a genetic sequence that regulates the expression of said polypeptide, which is operably linked to a reporter gene,
with a test agent; and
detecting a change in the level and/or functional activity of said polypeptide, or an expression product of said reporter gene, relative to a normal or reference level and/or functional activity in the absence of said test agent.

In another aspect, the invention resides in the use of an adhesin, or a biologically active fragment thereof, or a variant or derivative of these, as broadly described above, in crude or substantially purified form, to produce an antigen-binding molecule that is immuno-interactive with said adhesin, said biologically active fragment, said variant or said derivative, wherein said adhesin is obtainable from an organism which is capable of adhering to sperm and which correlates with the presence or risk of a condition selected from male infertility, an adverse pregnancy outcome or an adverse assisted reproductive technology (ART) outcome.

In yet another aspect, the invention provides an antigen-binding molecule so produced.

In still another aspect, the invention provides an antigen-binding molecule that is immuno-interactive with an adhesin that is expressible by an organism which is capable of adhering to sperm and which correlates with the presence or risk of a condition selected from male infertility, an adverse pregnancy outcome or an adverse assisted reproductive technology (ART) outcome.

According to another aspect of the invention, there is provided a method of detecting in a biological sample an adhesin, or a biologically active fragment thereof, or a variant or derivative of these, wherein said adhesin is expressible by an organism which is capable of adhering to sperm and which correlates with the presence or risk of a condition selected from male infertility, an adverse pregnancy outcome or an adverse assisted reproductive technology (ART) outcome, said method comprising:
contacting the sample with an antigen-binding molecule as broadly described above; and
detecting the presence of a complex comprising said antigen-binding molecule and said adhesin, fragment, variant or derivative in said contacted sample.

In yet another aspect, the invention contemplates a method for prognostic assessment of male infertility, adverse pregnancy outcome or adverse assisted reproductive technology (ART) outcome in a patient, comprising detecting in a biological sample obtained from said patient a gene encoding an adhesin or an expression product of said gene, wherein said adhesin is expressible by an organism which is capable of adhering to sperm and which correlates with the presence or risk of a condition selected from male infertility, an adverse pregnancy outcome or an adverse assisted reproductive technology (ART) outcome.

In yet another aspect of the invention, there is provided a method for masking an adhesin or for otherwise interfering with the binding of said adhesin to sperm, wherein said adhesin is expressible by an organism which is capable of adhering to sperm and which correlates with the presence or risk of a condition selected from male infertility, an adverse pregnancy outcome or an adverse assisted reproductive technology (ART) outcome, said method comprising contacting said adhesin with an antigen-binding molecule that is immuno-interactive with said adhesin.

In another aspect of the invention, there is provided a method for modulating adherence of an organism to sperm, wherein said organism is capable of adhering to sperm and correlates with the presence or risk of a condition selected from male infertility, an adverse pregnancy outcome or an adverse assisted reproductive technology (ART) outcome, said method comprising contacting said organism with an agent as broadly described above for a time and under conditions sufficient to modulate the level and/or functional activity of said adhesin.

The agent preferably decreases the level and/or functional activity of said adhesin.

In yet another aspect, the invention provides a composition for enhancing or otherwise improving male fertility, comprising an agent that reduces the level and/or functional activity of an adhesin that is expressible by an organism which is capable of adhering to sperm and which correlates with the presence or risk of male infertility, and optionally a pharmaceutically acceptable carrier.

In still yet another aspect, the invention provides a composition for enhancing the propensity for a favorable pregnancy outcome, comprising an agent that reduces the level and/or functional activity of an adhesin that is expressible by an organism which is capable of adhering to sperm and which correlates with the presence or risk of an adverse pregnancy outcome, and optionally a pharmaceutically acceptable carrier.

In another aspect, the invention extends to a composition for enhancing the propensity for a favorable assisted reproductive technology (ART) outcome, comprising an agent that reduces the level and/or functional activity of an adhesin that is expressible by an organism which is capable of adhering to sperm and which correlates with the presence or risk of an adverse ART outcome, and optionally a pharmaceutically acceptable carrier.

According to another aspect of the invention, there is provided a method for treatment and/or prophylaxis of male infertility, comprising administering to a patient in need of such treatment an effective amount of an agent that reduces the level and/or functional activity of an adhesin that is expressible by an organism which is capable of adhering to sperm and which correlates with the presence or risk of male infertility, in the presence or absence of a pharmaceutically acceptable carrier.

The invention, in yet another aspect, contemplates a method for treatment and/or prophylaxis of male infertility, comprising administering to the sperm of a patient in need of such treatment an effective amount of an agent that reduces the level and/or functional activity of an adhesin that is expressible by an organism which is capable of adhering to sperm and which correlates with the presence or risk of male infertility, in the presence or absence of a pharmaceutically acceptable carrier.

In still yet another aspect, the invention provides a method for enhancing the propensity for a favorable pregnancy outcome in a patient, comprising administering to a sperm donor of the patient an effective amount of an agent that reduces the level and/or functional activity of an adhesin that is expressible by an organism which is capable of adhering to sperm and which correlates with the presence or risk of an adverse pregnancy outcome, in the presence or absence of a pharmaceutically acceptable carrier.

In another aspect, the invention encompasses a method for enhancing the propensity for a favorable pregnancy outcome in a patient, comprising administering to the sperm of the patient's sperm donor an effective amount of an agent that reduces the level and/or functional activity of an adhesin that is expressible by an organism which is capable of adhering to sperm and which correlates with the presence or risk of an adverse pregnancy outcome, in the presence or absence of a pharmaceutically acceptable carrier.

In still yet another aspect, the invention extends to a method for enhancing the propensity for a favorable assisted reproductive technology (ART) outcome in a patient, comprising administering to the patient's sperm donor an effective amount of an agent that reduces the level and/or functional activity of an adhesin that is expressible by an organism which is capable of adhering to sperm and which correlates with the presence or risk of an adverse ART outcome, in the presence or absence of a pharmaceutically acceptable carrier.

In another aspect, the invention encompasses a method for enhancing the propensity for a favorable assisted reproductive technology (ART) outcome in a patient, comprising administering to the sperm of the patient's sperm donor an effective amount of an agent that reduces the level and/or functional activity of an adhesin that is expressible by an organism which is capable of adhering to sperm and which correlates with the presence or risk of an adverse ART outcome.

According to another aspect, the invention extends to an immunopotentiating composition for eliciting the production of elements that specifically bind to an organism which is capable of adhering to sperm and which correlates with the presence or risk of a condition selected from male infertility, an adverse pregnancy outcome or an adverse assisted reproductive technology (ART) outcome, said composition comprising a proteinaceous molecule selected from an isolated adhesin of said organism, a biologically active fragment of said adhesin, a variant of said adhesin, a variant of said biologically active fragment, a derivative of said adhesin, a derivative of said biologically active fragment, and a derivative of said variant, and/or comprising a vector including a polynucleotide encoding said proteinaceous molecule and operably linked to a regulatory polynucleotide, wherein said composition optionally further comprises a pharmaceutically acceptable carrier or adjuvant.

In yet another aspect, the invention encompasses a method for treatment and/or prophylaxis of male infertility, comprising administering to a patient in need of such treatment an immunogenically effective amount of the immunopotentiating composition as broadly described above.

In still yet another aspect, the invention features a method for treatment and/or prophylaxis of an adverse pregnancy outcome in a patient, comprising administering to the patient's sperm donor an immunogenically effective amount of the immunopotentiating composition as broadly described above.

In a further aspect, the invention envisions a method for treatment and/or prophylaxis of an adverse assisted reproductive technology (ART) outcome in a patient, comprising administering to the patient's sperm donor an immunogenically effective amount of the immunopotentiating composition as broadly described above.

The invention also encompasses the use of the adhesin, fragment, variant or derivative as well as the modulatory agents as broadly described above in the study, and modulation of male infertility, pregnancy or assisted reproductive technology (ART) outcome.

### DETAILED DESCRIPTION OF THE INVENTION

1. Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

The articles “a” and “an” are used herein to refer to one or to more than one (i.e. to at least one) of the grammatical object of the article. By way of example, “an element” means one element or more than one element.

By “agent” is meant a naturally occurring or synthetically produced molecule which interacts either directly or indirectly with a target member, the level and/or functional activity of which are to be modulated.

“Amplification product” refers to a nucleic acid product generated by nucleic acid amplification techniques.

By “antigen-binding molecule” is meant a molecule that has binding affinity for a target antigen. It will be understood that this term extends to immunoglobulins, immunoglobulin fragments and non-immunoglobulin derived protein frameworks that exhibit antigen-binding activity.

As used herein, the term “binds specifically”, “specifically immuno-interactive” and the like refers to antigen-binding molecules that bind, or are otherwise immuno-interactive with, the polypeptide or polypeptide fragments of the invention but do not significantly bind to, or do not otherwise specifically immuno-interact with, homologous prior art polypeptides.

By “biologically active fragment” is meant a fragment of a full-length parent polypeptide which fragment retains the activity of the parent polypeptide. A biologically active fragment will therefore adhere to sperm, or elicit an immunogenic response to produce elements (e.g., antigen-binding molecules) that specifically bind to the parent polypeptide. As used herein, the term “biologically active fragment” includes deletion mutants and small peptides, for example of at least 8, preferably at least 10, more preferably at least 15, even more preferably at least 20 and even more preferably at least 30 contiguous amino acids, which comprise the above activities. Peptides of this type may be obtained through the application of standard recombinant nucleic acid techniques or synthesized using conventional liquid or solid phase synthesis techniques. For example, reference may be made to solution synthesis or solid phase synthesis as described, for example, in Chapter 9 entitled “Peptide Synthesis” by Atherton and Shephard which is included in a publication entitled “Synthetic Vaccines” edited by Nicholson and published by Blackwell Scientific Publications. Alternatively, peptides can be produced by digestion of a polypeptide of the invention with proteinases such as endoLys-C, endoArg-C, endoGlu-C and staphylococcus V8-protease. The digested fragments can be purified by, for example, high performance liquid chromatographic (HPLC) techniques.

The term “biological sample” as used herein refers to a sample that may be extracted, untreated, treated, diluted or concentrated from an animal. The biological sample may be selected from the group consisting of whole blood, serum, plasma, saliva, urine, sweat, ascitic fluid, peritoneal fluid, synovial fluid, amniotic fluid, cerebrospinal fluid, skin biopsy, and the like. Preferably, the biological sample is selected from amniotic fluid, seminiferous tubule fluid (e.g., in vasectomized men), semen, vaginal secretions, endocervical secretions, respiratory secretions, endometrial lining, fallopian tube washings, follicular fluid, spermatozoa, endocervical cells, placental tissue, endometrial biopsies, chorioamnion, seminiferous tubule tissue, ovarian tissue, oocytes, embryos singles cells biopsied from embryos, fallopian tube tissue.

Throughout this specification, unless the context requires otherwise, the words “comprise”, “comprises” and “comprising” will be understood to imply the inclusion of a stated step or element or group of steps or elements but not the exclusion of any other step or element or group of steps or elements.

By “corresponds to” or “corresponding to” is meant a polynucleotide (a) having a nucleotide sequence that is substantially identical or complementary to all or a portion of a reference polynucleotide sequence or (b) encoding an amino acid sequence identical to an amino acid sequence in a peptide or protein. This phrase also includes within its scope a peptide or polypeptide having an amino acid sequence that is substantially identical to a sequence of amino acids in a reference peptide or protein.

By “derivative” is meant a polypeptide that has been derived from the basic sequence by modification, for example by conjugation or complexing with other chemical moieties or by post-translational modification techniques as would be understood in the art. The term “derivative” also includes within its scope alterations that have been made to a parent sequence including additions, or deletions that provide for functionally equivalent molecules. Accordingly, the term derivative encompasses molecules that will adhere to sperm, and the elicitation of an immunogenic response to produce elements (e.g., antigen-binding molecules) that specifically bind to the parent adhesin.

By “effective amount” in the context of treating or preventing a condition described herein, is meant the administration of that amount of modulatory agent, which modulates the expression of a gene encoding an adhesin as broadly described above or the level and/or functional activity of an expression product of that gene, or that amount of immunopotentiating agent as broadly described above, to an individual in need thereof, either in a single dose or as part of a series, that is effective for the treatment or prevention of that condition. The effective amount will vary depending upon the health and physical condition of the individual to be treated, the taxonomic group of individual to be treated, the formulation of the composition, the assessment of the medical situation, and other relevant factors. It is expected that the amount will fall in a relatively broad range that can be determined through routine trials.

As used herein, the term “function” refers to a biological, enzymatic, or therapeutic function.

“Homology” refers to the percentage number of amino acids that are identical or constitute conservative substitutions as defined in Table A infra. Homology may be determined using sequence comparison programs such as GAP (Deveraux et al. 1984). In this way, sequences of a similar or substantially different length to those cited herein might be compared by insertion of gaps into the alignment, such gaps being determined, for example, by the comparison algorithm used by GAP. “Hybridization” is used herein to denote the pairing of complementary nucleotide sequences to produce a DNA-DNA hybrid or a DNA-RNA hybrid. Complementary base sequences are those sequences that are related by the base-pairing rules. In DNA, A pairs with T and C pairs with G. In RNA U pairs with A and C pairs with G. In this regard, the terms “match” and “mismatch” as used herein refer to the hybridization potential of paired nucleotides in complementary nucleic acid strands. Matched nucleotides hybridize efficiently, such as the classical A-T and G-C base pair mentioned above. Mismatches are other combinations of nucleotides that do not hybridize efficiently.

Reference herein to “immuno-interactive” includes reference to any interaction, reaction, or other form of association between molecules and in particular where one of the molecules is, or mimics, a component of the immune system.

By “isolated” is meant material that is substantially or essentially free from components that normally accompany it in its native state. For example, an “isolated polynucleotide”, as used herein, refers to a polynucleotide, which has been purified from the sequences which flank it in a naturally occurring state, e.g., a DNA fragment which has been removed from the sequences which are normally adjacent to the fragment.

By “modulating” is meant increasing or decreasing, either directly or indirectly, the level and/or functional activity of a target molecule. For example, an agent may indirectly modulate the said level/activity by interacting with a molecule other than the target molecule. In this regard, indirect modulation of a gene encoding a target polypeptide includes within its scope modulation of the expression of a first nucleic acid molecule, wherein an expression product of the first nucleic acid molecule modulates the expression of a nucleic acid molecule encoding the target polypeptide.

By “obtained from” is meant that a sample such as, for example, a nucleic acid extract or polypeptide extract is isolated from, or derived from, a particular source of the host. For example, the extract may be obtained from a tissue or a biological fluid isolated directly from the host.

The term “oligonucleotide” as used herein refers to a polymer composed of a multiplicity of nucleotide units (deoxyribonucleotides or ribonucleotides, or related structural variants or synthetic analogues thereof) linked via phosphodiester bonds (or related structural variants or synthetic analogues thereof). Thus, while the term “oligonucleotide” typically refers to a nucleotide polymer in which the nucleotides and linkages between them are naturally occurring, it will be understood that the term also includes within its scope various analogues including, but not restricted to, peptide nucleic acids (PNAs), phosphoramidates, phosphorothioates, methyl phosphonates, 2-O-methyl ribonucleic acids, and the like. The exact size of the molecule may vary depending on the particular application. An oligonucleotide is typically rather short in length, generally from about 10 to 30 nucleotides, but the term can refer to molecules of any length, although the term “polynucleotide” or “nucleic acid” is typically used for large oligonucleotides.

By “operably linked”, “operably connected”, “operable linkage” and the like is meant a linkage of polynucleotide elements in a functional relationship. A nucleic acid is “operably linked” when it is placed into a functional relationship with another nucleic acid sequence. For instance, a promoter or enhancer is operably linked to a coding sequence if it affects the transcription of the coding sequence. “Operably connecting” a promoter to a polynucleotide is meant placing the polynucleotide (e.g., protein encoding polynucleotide or other transcript) under the regulatory control of a promoter, which then controls the transcription and optionally translation of that polynucleotide. In the construction of heterologous promoter/structural gene combinations, it is generally preferred to position a promoter or variant thereof at a distance from the transcription start site of the polynucleotide, which is approximately the same as the distance between that promoter and the gene it controls in its natural setting; i.e.: the gene from which the promoter is derived. As is known in the art, some variation in this distance can be accommodated without loss of function.

The term “patient” refers to patients of human or other animal and includes any individual it is desired to examine or treat using the methods of the invention. However, it will be understood that “patient” does not imply that symptoms are present. Suitable animals that fall within the scope of the invention include, but are not restricted to, primates, livestock animals (e.g. sheep, cows, horses, donkeys, pigs), laboratory test animals (e.g. rabbits, mice, rats, guinea pigs, hamsters), companion animals (e.g. cats, dogs) and captive wild animals (e.g. foxes, deer, dingoes, avians and reptiles). Preferably, the patient is a mammal, more preferably a primate and still more preferably a human.

By “pharmaceutically-acceptable carrier” is meant a solid or liquid filler, diluent or encapsulating substance that may be safely used in topical or systemic administration.

The term “polynucleotide” or “nucleic acid” as used herein designates mRNA, RNA, cRNA, cDNA or DNA. The term typically refers to oligonucleotides greater than 30 nucleotides in length. Polynucleotide sequences are understood to encompass complementary strands as well as alternative backbones described herein.

The terms “polynucleotide variant” and “variant” refer to polynucleotides displaying substantial sequence identity with a reference polynucleotide sequence or polynucleotides that hybridize with a reference sequence under stringent conditions that are defined hereinafter. These terms also encompasses polynucleotides in which one or more nucleotides have been added or deleted, or replaced with different nucleotides. In this regard, it is well understood in the art that certain alterations inclusive of mutations, additions, deletions and substitutions can be made to a reference polynucleotide whereby the altered polynucleotide retains the biological function or activity of the reference polynucleotide. The terms “polynucleotide variant” and “variant” also include naturally occurring allelic variants.

“Polypeptide”, “peptide” and “protein” are used interchangeably herein to refer to a polymer of amino acid residues and to variants and synthetic analogues of the same. Thus, these terms apply to amino acid polymers in which one or more amino acid residues is a synthetic non-naturally occurring amino acid, such as a chemical analogue of a corresponding naturally occurring amino acid, as well as to naturally-occurring amino acid polymers.

The term “polypeptide variant” refers to polypeptides which vary from a reference polypeptide by the addition, deletion or substitution of at least one amino acid. It is well understood in the art that some amino acids may be changed to others with broadly similar properties without changing the nature of the activity of the polypeptide. Accordingly, polypeptide variants as used herein encompass polypeptides that have similar activities to a parent adhesin polypeptide. Preferred variant polypeptides comprise conservative amino acid substitutions. Exemplary conservative substitutions in a polypeptide may be made according to Table A:

TABLE A

| Original Residue | Exemplary Substitutions |
|---|---|
| Ala | Ser |
| Arg | Lys |
| Asn | Gln, His |
| Asp | Glu |
| Cys | Ser |
| Gln | Asn |
| Glu | Asp |
| Gly | Pro |
| His | Asn, Gln |
| Ile | Leu, Val |
| Leu | Ile, Val |
| Lys | Arg, Gln, Glu |
| Met | Leu, Ile, |
| Phe | Met, Leu, Tyr |
| Ser | Thr |
| Thr | Ser |
| Trp | Tyr |
| Tyr | Trp, Phe |
| Val | Ile, Leu |

Substantial changes in function are made by selecting substitutions that are less conservative than those shown in Table A. Other replacements would be non-conservative substitutions and relatively fewer of these may be tolerated. Generally, the substitutions which are likely to produce the greatest changes in a polypeptide's properties are those in which (a) a hydrophilic residue (e.g., Ser or Asn) is substituted for, or by, a hydrophobic residue (e.g., Ala, Leu, Ile, Phe or Val); (b) a cysteine or proline is substituted for, or by, any other residue; (c) a residue having an electropositive side chain (e.g., Arg, His or Lys) is substituted for, or by, an electronegative residue (e.g., Glu or Asp) or (d) a residue having a smaller side chain (e.g., Ala, Ser) or no side chain (e.g., Gly) is substituted for, or by, one having a bulky side chain (e.g., Phe or Trp).

By “primer” is meant an oligonucleotide which, when paired with a strand of DNA, is capable of initiating the synthesis of a primer extension product in the presence of a suitable polymerizing agent. The primer is preferably single-stranded for maximum efficiency in amplification but may alternatively be double-stranded. A primer must be sufficiently long to prime the synthesis of extension products in the presence of the polymerzation agent. The length of the primer depends on many factors, including application, temperature to be employed, template reaction conditions, other reagents, and source of primers. For example, depending on the complexity of the target sequence, the oligonucleotide primer typically contains 15 to 35 or more nucleotides, although it may contain fewer nucleotides. Primers can be large polynucleotides, such as from about 200 nucleotides to several kilobases or more. Primers may be selected to be “substantially complementary” to the sequence on the template to which it is designed to hybridize and serve as a site for the initiation of synthesis. By “substantially complementary”, it is meant that the primer is sufficiently complementary to hybridize with a target nucleotide sequence. Preferably, the primer contains no mismatches with the template to which it is designed to hybridize but this is not essential. For example, non-complementary nucleotides may be attached to the 5′ end of the primer, with the remainder of the primer sequence being complementary to the template. Alternatively, non-complementary nucleotides or a stretch of non-complementary nucleotides can be interspersed into a primer, provided that the primer sequence has sufficient complementarity with the sequence of the template to hybridize therewith and thereby form a template for synthesis of the extension product of the primer.

“Probe” refers to a molecule that binds to a specific sequence or sub-sequence or other moiety of another molecule. Unless otherwise indicated, the term “probe” typically refers to a polynucleotide probe that binds to another nucleic acid, often called the “target nucleic acid”, through complementary base pairing. Probes may bind target nucleic acids lacking complete sequence complementarity with the probe, depending on the stringency of the hybridization conditions. Probes can be labelled directly or indirectly.

The term “recombinant polynucleotide” as used herein refers to a polynucleotide formed in vitro by the manipulation of nucleic acid into a form not normally found in nature. For example, the recombinant polynucleotide may be in the form of an expression vector. Generally, such expression vectors include transcriptional and translational regulatory nucleic acid operably linked to the nucleotide sequence.

By “recombinant polypeptide” is meant a polypeptide made using recombinant techniques, i.e., through the expression of a recombinant polynucleotide.

By “reporter molecule” as used in the present specification is meant a molecule that, by its chemical nature, provides an analytically identifiable signal that allows the detection of a complex comprising an antigen-binding molecule and its target antigen. The term “reporter molecule” also extends to use of cell agglutination or inhibition of agglutination such as red blood cells on latex beads, and the like.

Terms used to describe sequence relationships between two or more polynucleotides or polypeptides include “reference sequence”, “comparison window”, “sequence identity”, “percentage of sequence identity” and “substantial identity”. A “reference sequence” is at least 12 but frequently 15 to 18 and often at least 25 monomer units, inclusive of nucleotides and amino acid residues, in length. Because two polynucleotides may each comprise (1) a sequence (i.e., only a portion of the complete polynucleotide sequence) that is similar between the two polynucleotides, and (2) a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window” refers to a conceptual segment of typically 12 contiguous residues that is compared to a reference sequence. The comparison window may comprise additions or deletions (i.e., gaps) of about 20% or less as compared to the reference sequence (which does not comprise additions or deletions) for optimal alignment of the two sequences. Optimal alignment of sequences for aligning a comparison window may be conducted by computerized implementations of algorithms (GAP, BESTFIT, FASTA, and TFASTA in the Wisconsin Genetics Software Package Release 7.0, Genetics Computer Group, 575 Science Drive Madison, Wis., USA) or by inspection and the best alignment (i.e., resulting in the highest percentage homology over the comparison window) generated by any of the various methods selected. Reference also may be made to the BLAST family of programs as for example disclosed by Altschul et al., 1997. A detailed discussion of sequence analysis can be found in Unit 19.3 of Ausubel et al., “Current Protocols in Molecular Biology”, John Wiley & Sons Inc, 1994-1998, Chapter 15.

The term “sperm donor” as used herein refers in its broadest sense to any donor whose sperm is used, or desired to be used, for fertilization whether by natural or artificial means and shall include the patient's sexual partner or any other individual.

The term “sequence identity” as used herein refers to the extent that sequences are identical on a nucleotide-by-nucleotide basis or an amino acid-by-amino acid basis over a window of comparison. Thus, a “percentage of sequence identity” is calculated by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity. For the purposes of the present invention, “sequence identity” will be understood to mean the “match percentage” calculated by the DNASIS computer program (Version 2.5 for windows; available from Hitachi Software engineering Co., Ltd., South San Francisco, Calif., USA) using standard defaults as used in the reference manual accompanying the software.

“Stringency” as used herein, refers to the temperature and ionic strength conditions, and presence or absence of certain organic solvents, during hybridization and washing procedures. The higher the stringency, the higher will be the degree of complementarity between immobilized target nucleotide sequences and the labelled probe polynucleotide sequences that remain hybridized to the target after washing.

“Stringent conditions” refers to temperature and ionic conditions under which only nucleotide sequences having a high frequency of complementary bases will hybridize. The stringency required is nucleotide sequence dependent and depends upon the various components present during hybridization and subsequent washes, and the time allowed for these processes. Generally, in order to maximise the hybridization rate, non-stringent hybridization conditions are selected; about 20° to about 25° C. lower than the thermal melting point (Tₘ). The Tₘ is the temperature at which 50% of specific target sequence hybridizes to a perfectly complementary probe in solution at a defined ionic strength and pH. Generally, in order to require at least about 85% nucleotide complementarity of hybridized sequences, highly stringent washing conditions are selected to be about 5° to about 15° C. lower than the Tₘ. In order to require at least about 70% nucleotide complementarity of hybridized sequences, moderately stringent washing conditions are selected to be about 15° to about 30° C. lower than the Tₘ. Highly permissive (low stringency) washing conditions may be as low as 50° C. below the Tₘ, allowing a high level of mis-matching between hybridized sequences. Those skilled in the art will recognise that other physical and chemical parameters in the hybridization and wash stages can also be altered to affect the outcome of a detectable hybridization signal from a specific level of homology between target and probe sequences.

By “vector” is meant a nucleic acid molecule, preferably a DNA molecule derived, for example, from a plasmid, bacteriophage, or plant virus, into which a nucleic acid sequence may be inserted or cloned. A vector preferably contains one or more unique restriction sites and may be capable of autonomous replication in a defined host cell including a target cell or tissue or a progenitor cell or tissue thereof, or be integrable with the genome of the defined host such that the cloned sequence is reproducible. Accordingly, the vector may be an autonomously replicating vector, i.e., a vector that exists as an extrachromosomal entity, the replication of which is independent of chromosomal replication, e.g., a linear or closed circular plasmid, an extrachromosomal element, a minichromosome, or an artificial chromosome. The vector may contain any means for assuring self-replication. Alternatively, the vector may be one which, when introduced into the host cell, is integrated into the genome and replicated together with the chromosome(s) into which it has been integrated. A vector system may comprise a single vector or plasmid, two or more vectors or plasmids, which together contain the total DNA to be introduced into the genome of the host cell, or a transposon. The choice of the vector will typically depend on the compatibility of the vector with the host cell into which the vector is to be introduced. The vector may also include a selection marker such as an antibiotic resistance gene that can be used for selection of suitable transformants. Examples of such resistance genes are well known to those of skill in the art.

2. Ureaplasmas of the Invention

The present invention is predicated in part on the determination that certain organisms, which adhere more strongly to spermatozoa than other organisms, are more likely to impact adversely on male fertility and on pregnancy outcomes, including ART pregnancy outcomes such as, but not limited to, reduced embryonic development, reduced implantation rate, reduced fertilization rate, reduced clinical pregnancy rate, reduced viable pregnancy rate, reduced blastocyst culture rate and increased miscarriage rate. Other adverse pregnancy outcomes associated with the organisms of the invention include, but are not limited to, miscarriage, pre-term delivery, premature onset of labor, prolonged rupture of membranes, neonatal morbidity and mortality.

Accordingly, the invention broadly contemplates methods of ascertaining the propensity (a) for male fertility, or (b) an adverse pregnancy outcome or (c) for an adverse ART outcome, by detecting a strongly adherent ureaplasma according to the invention in a biological sample obtained from a patient or from a patient's sperm donor. The invention also contemplates improving male fertility or enhancing the propensity of a favorable pregnancy outcome, or for enhancing the propensity of a favorable ART outcome by treating a patient, or the sperm of a patient, with an agent that reduces the level and/or functional activity of an adhesin from an organism as broadly described above or that treats or prevents infection by organisms expressing said adhesin, as described hereinafter.

Accordingly, the invention provides, in one aspect, a method for detecting a reproductive function-modulating organism associated with a condition selected from male infertility, an adverse pregnancy outcome or an adverse assisted reproductive technology (ART) outcome, or a higher risk of said condition. The method comprises detecting an organism which is capable of adhering to sperm and which correlates with the presence or risk of said condition.

The correlation with the presence or risk of the condition is preferably made if the organism is capable of remaining adherent to sperm after washing a sample comprising organism-positive sperm to permit removal or separation of non-sperm substances from the sample. For example, a positive correlation is made if the washed sperm sample comprises at least 0.01%, preferably at least 0.1%, more preferably at least 1%, even more preferably at least 2%, even more preferably at least 4%, even more preferably at least 10%, even more preferably at least 20% and still even more preferably at least 50% of the total number of organisms present in said sperm sample before washing.

The washing is preferably carried out using wash conditions that protect one or more viability properties of the sperm such as but not limited to sperm motility, sperm numbers, percentage of living sperm recovered, sperm membrane function, sperm penetration rate, sperm in vitro fertilization rate and/or oocyte development following fertilization. For example, a sperm sample can be washed using a wash solution, which is capable of removing non-sperm substances, or non-motile sperm or sperm fragments, from the sample, while also maintaining sperm viability properties during washing. Components which can be removed from a sperm sample by washing in a solution include bacteria, seminal plasma, proteins, antibodies, white blood cells, red blood cells, freezing extender agents, such as egg yolk, sperm debris, such as non-motile, or non-living sperm or sperm fragments, as well as culture media and media supplements. Alternatively, or additionally, sperm can be separated from non-sperm substances and/or non viable sperm by allowing the motile sperm to swim away from the debris (sperm swim-up), by centrifuging the sperm through a gradient and collecting a pellet of live sperm (washing), or by passing the sperm through a column that binds the dead or unhealthy sperm. Suitable methods and compositions for washing and separating sperm are described for example by Trounson and Gardner (Handbook of In Vitro Fertilization, CRC Press, Boca Raton, 1994, pp. 46-50). Alternatively, reference may be made: to Bongso et al., (1989, Fertility and Sterility, 51: 850-854) who describe a method for improving concentration and motility of sperm by Ficoll separation; to Ericsson (U.S. Pat. No. 4,007,087) who discloses fractionating sperm in a medium including soluble materials such as proteins, peptides and dextran; to Shrimpton (U.S. Pat. No. 4,327,177) who discloses the separation of sperm by density in a nutrient media derived from mammalian milk; to Shrimpton (U.S. Pat. No. 4,605,558) who discloses a method of separating X and Y sperm in a density gradient and an osmolality gradient in a medium derived from milk; to Ellington et al. (U.S. Pat. No. 6,171,778) who describe the use of an arabinogalactan for separating motile sperm; and to Zavos et al. (U.S. Pat. No. 5,908,380) who describe a compartmentalized swim-up column for the swimming-up of spermatozoa, at desired levels of dilution in a media, and a swimming down of spermatozoa into compartments within the column, as well as a method for the harvesting of semen samples having particular sperm morphology, motility, progressive motility, speed, sperm concentration, fertilization potential, and a sex ratio. Preferred separation methods include gradient centrifugation methods and sperm swim-up protocols, as for example described in Examples 3 and 5.

In a preferred embodiment, the wash conditions are characterised in that they permit the continuation of adherence of Ureaplasma parvum serotype 6 to said sperm but do not permit the continuation of adherence of U. parvum serotype 1 to said sperm. In a preferred embodiment, the washing is carried out using a sperm swim-up technique. In another preferred embodiment, the washing is carried out using gradient centrifugation. As used herein, the phrase “conditions which permit the continuation of adherence” shall mean separation conditions that permit the continuation of adherence to a sperm sample of at least 90%, preferably at least 80%, more preferably at least 70%, even more preferably at least 60%, even more preferably at least 50%, and still even more preferably at least 40% of ureaplasmas which adhered to the sperm sample before separation. By contrast, the phrase “conditions which do not permit the continuation of adherence” shall mean separation conditions that permit the continuation of adherence to a sperm sample of no more than 30%, preferably no more than 25%, more preferably no more than 20%, even more preferably no more than 15%, and still even more preferably no more than 10% of ureaplasmas which adhered to the sperm sample before separation.

Any method for qualitatively or quantitatively determining sperm viability properties is contemplated by the present invention. For example, sperm numbers in a suspension can be determined by manual or computerized methods. Using computerized methods, a sperm suspension is applied to a counting chamber available in the art such as a Makler™ (Fertility Technology, Natick, Mass.) counting chamber, and the number of sperm is counted, which is equal the number of sperm/mL in the original suspension. Sperm morphology or shape is determined, for example, by smearing 10 μL of a sperm sample onto a slide and staining with a differential stain such as Wright Giemsa at 0.1% (w/v) for 30 min or by placing 5 μL of sperm onto a pre-stained slide for morphology such as “Testsimplets” (Daintree Industries), allowing the slide to sit for 15 minutes. Sperm then are observed under a microscope by oil immersion and categorised as to normal or abnormal shapes (morphology), as described in Kruger et al. (1987, Urology 30: 248). Motility of sperm is expressed as the total percent of motile sperm, or the speed of the sperm that are motile and can be determined using methods available in the art, such as by subjective visual determination using a phase contrast microscope, or using a computer automated semen analyser. Using phase contrast microscopy, the sample is analyzed visually to group sperm into total percent motile (swimming), and total percent progressively motile (swimming forward), or the speed of the sperm which are progressively motile, i.e., fast, medium, or slow. Using a computer, the track speed of individual sperm is analyzed. Data is expressed as the percent motile, as well as the mean path velocity and track speed of sperm in the sample. Sperm viability as a measurement of the percentage of living sperm is determined by membrane exclusion stains available in the art. Sperm membrane function of live sperm is tested by placing sperm into a low salt (hypo-osmotic) solution. This causes sperm with healthy membranes to pump salt out of the cell, and causes the membranes of the sperm to shrink as the cell grows smaller. The sperm tail then curls inside this tighter membrane. Sperm with a curled tail are the sperm which are healthy and have functional membranes. The number of sperm with a curled tail then is expressed as a percent of the total number of sperm present. To assay sperm penetration, the ability of capacitated sperm to penetrate a dead zona free hamster egg is measured. Sperm in vitro fertilization rates are determined by measuring the percent of oocytes fertilized in vitro using methods available in the art. The capacitated sperm sample is incubated with oocytes, or a single sperm is injected into an oocyte and at the end of the incubation, the percentage of oocytes fertilized is determined, or the fertilized oocytes are left in culture, division occurs and the number of cleaving embryos is determined.

From the foregoing, it will be understood that the invention contemplates any organism that infects an animal, especially a mammal, more especially a primate and even more especially a human, and remains adherent to sperm after subjecting the sperm to washing conditions as for example described above. Such organisms are referred to hereinafter as “adherovars”. An adherovar is preferably selected from the class Mollicutes, especially from the order Mycoplasmatales, more especially from the family Mycoplasmataceae, even more especially from the genus Mycoplasma, and still even more especially from the genus Ureaplasma. In a preferred embodiment, the adherovar is selected from the genus Mycoplasma, which includes, but is not limited to, Candidatus M. kahanei, Candidatus M. ravipulmonis, Eperythrozoon ovis, Haemobartonella canis, M. adleri, M. agalactiae, M. agassizii, M. alkalescens, M. alligatoris, M. alvi, M. anatis, M. anseris, M. arginini, M. arthritidis, M. auris, M. bovigenitalium, M. bovirhinis, M. bovis, M. bovoculi, M. buccale, M. buteonis, M. californicum, M. canadense, M. canis, M. caviae, M. cavipharyngis, M. citelli, M. cloacale, M. collis, M. columbinasale, M. columbinum, M. columborale, M. conjunctivae, M. corogypsi, M. cottewii, M. cricetuli, M. crocodyli, M. cynos, M. dispar, M. edwardii, M. elephantis, M. equigenitalium, M. equirhinis, M. erythrodidelphis, M. falconis, M. fastidiosum, M. faucium, M. felifaucium, M. feliminutum, M. felis, M. fermentans, M. flocculare, M. gallinaceum, M. gallinarum, M. gallisepticum, M. gallopavonis, M. gateae, M. genitalium, M. glycophilum, M. gypis, M. haemofelis, M. haemomuris, M. haemosuis, M. hominis, M. hyopharyngis, M. hyopneumoniae, M. hyorhinis, M. hyosynoviae, M. imitans, M. indiense, M. iners, M. iowae, M. lagogenitalium, M. leonicaptivi, M. leopharyngis, M. lipofaciens, M. lipophilum, M. maculosum, M. meleagridis, M. microti, M. moatsii, M. mobile, M. molare, M. monodon, M. muris, M. niustelae, M. neurolyticum, M. opalescens, M. orale, M. ovipneumoniae, M. oxoniensis, M. penetrans, M. phocicerebrale, M. phocidae, M. phocirhin is, M. pirum, M. pneumoniae, M. pneumophila, M. primatum, M. pullorum, M. pulmonis, M. salivariuni, M. simbae, M. spennatophilum, M. spumans, M. sturni, M. sualvi, M. subdolum, M. synoviae, M. testudinis, M. verecundum, M. wenyonii, M. yeatsii, M. zalophus, M. sp., M. sp. ‘bovine group 7’, M. sp. 94630, M. sp. serogroup L. In a preferred embodiment of this type, the adherovar is a subtype of a strain or serotype of M. hominis.

In a preferred embodiment, the adherovar is a ureaplasma. For example, the ureaplasma may be selected from a strain or serotype of the genus Ureaplasma (e.g., a species of U. canigenitalium, U. cati, U. diversum, U. felinum, U. gallorale, U. parvum, or U. urealyticum). In a preferred embodiment, the adherovar is a subtype of a strain or serotype (i.e., serovar) of U. parvum or U. urealyticuni. In an especially preferred embodiment of this type, the ureaplasma is a subtype of U. parvum serotype 6.

Detection of an adherovar can be carried out at the functional or physical level as known in the art. For example, in the case of ureaplasmas, reference may be made to standard microbiological techniques that generally identify ureaplasma by observing the hydrolysis of urea. These techniques usually involve inoculating both a complex broth medium and an agar medium containing urea and other nutrients with a freshly obtained specimen (Brunner et al., 1983, Yale J. Biol. Med. 56: 545; Shephard and Lunceford, 1978, J. Clin. Micro. 8: 566-574). References concerning physical detection of ureaplasma include the following: Stemke and Robertson (1985, Diagn. Microbiol. Infect. Dis. 31: 311) who disclose fourteen serotypes of U. urealyticum; Harasawa et al. (1990, Abstract S30-6 UIMS Meeting, Osaka Japan), Robertson et al. (1993, J. Clin. Microbiol. 31: 824), Hammond et al. (1991, Abstract D17. Session 60, American Society for Microbiology General Meeting), who teach that the fourteen serotypes can be divided into at least two subspecies (“biotypes”) and Kong et al. 2000, Int J Syst Evol Microbiol 50 (5): 1921-1929) and Robertson et al. 2002, Int J Syst Evol Microbiol. 52 (2):587-97, who disclose that the two biotypes are separated into 2 different species Biovar 1 (parvo biovar), which is now U. parvum serovars 1, 3, 6, and 14 and Biovar 2, (T960 biovar), which is now U. urealyticum serovar 2, 4, 5, 7-13, based upon restriction fragment length polymorphism (RFLP) of U. urealyticum genomic or based upon rRNA sequences; Roberts et al. (1987, Israel J. Med. Sci. 23: 618) who describe the use of whole chromosomal DNA probes for detection of ureaplasma in genital specimens; Ohse and Gobel (1987, Israel J. Med. Sci. 23: 352) who describe hybridization of U. urealyticum rRNA genes to cloned DNA of the E. coli rRNA operon; Gobel and Stanbridge (EP-A-0 250 662) who mention biological probes for detecting Mycoplasmas or prokaryotes in general, or specific Mycoplasma and eubacterial species; Gonzales et al. (1991, American Society for Microbiology Annual Meeting, Abstract D-16) who mention a method to detect ureaplasma using a DNA probe directed to rRNA; Lee et al. (1992, Arthritis and Rheumatism 35: 43) and Willoughby et al. (1991, Infection and Immunity 59: 2463), who describe a procedure for detecting the U. urealyticum urease gene utilising PCR; Brogan et al. (1992, Molecular and Cellular Probes 6: 411) who describe the amplification of a 186 base pair genomic U. urealyticum DNA fragment; Robertson et al. (1993, supra) who describe a technique involving the polymerase chain reaction (PCR) using biotype specific primers to 16S rRNA gene sequences to distinguish two U. urealyticum biotypes; and Hogan et al. (U.S. Pat. No. 6,093,538) who teach hybridization probes which can distinguish the genus Ureaplasma, including clinically significant U. urealyticum serotypes, from their known closest phylogenetic neighbours (Mycoplasma) and from other microorganism inhabitants of the human urogenital tract and which function by hybridising to target U. urealyticum rRNA and/or rRNA gene sequences under stringent hybridization assay conditions. Preferably, an ureaplasma is detected using the nested PCR technique disclosed herein, which distinguishes strain or serotypes of ureaplasma based on the nucleotide sequence of their respective multiple band antigen (mba) genes. Alternatively, the PCR method of Teng et al. (1994, J. Clin. Micro 32: 1464-1469) may be employed, which uses primers based on the mba gene, and which distinguishes species of ureaplasma by the size of the amplified fragment. 403-404 bp for U. parvum, 448 bp for U. urealyticum.

The positive detection of an adherovar is indicative that a patient has a propensity (a) for male fertility, or (b) for an adverse pregnancy outcome or (b) for an adverse ART outcome.

3. Isolation of Adhesin

From the foregoing, it is believed that the enhanced adherence to, or affinity for, spermatozoa displayed by the adherovars of the invention is mediated by one or more adhesins present on their surface. Accordingly, in another aspect, the invention contemplates an isolated adhesin or a biologically active fragment thereof, or a variant or derivative of these, wherein the adhesin is obtainable from an organism which is capable of adhering to sperm and which correlates with the presence or risk of a condition selected from male infertility, an adverse pregnancy outcome or an adverse assisted reproductive technology (ART) outcome.

Any method suitable for detecting protein—protein interactions may be employed for identifying adhesin proteins. Among the traditional methods which may be employed are co-immunoprecipitation, crosslinking and co-purification through gradients or chromatographic columns. For example, an antigen-binding molecule (e.g., a monoclonal antibody), which is immuno-interactive specifically with an adherovar according to the invention, can be used to isolate the adhesin from a membrane fraction of the adherovar.

Alternatively, one can take advantage of the property that the adhesins of many pathogens typically bind to specific carbohydrate moieties, enabling colonization and infection. Ureaplasmas are also known to be capable of binding carbohydrate moieties. These carbohydrate moieties may be present in the form of either glycolipids or glycoproteins. For example, many pathogens recognise gangliotriaosylceramide (GalNAcβ1-4galβ1-4glc cer [Gg₃]), gangliotetraosylceramide (galβ1-3galNAc.β1-4galβ1-4glc cer [Gg₄]), sulfogalactosylceramide (SGC), sulfatoxygalactosylceramide (SGC) sulfogalastosylglycerolipid (SGG) and phosphatidylethanolamine (PE). Thus, an affinity matrix comprising a carbohydrate (glycolipid) moiety can be prepared and used to isolate an adhesin from an adherovar of interest as, for example, disclosed by Lingwood (U.S. Pat. No. 6,218,147). In a preferred embodiment, the adhesin, which is preferably a ureaplasma adhesin, is isolated using SGG and/or SGC in concert for example with a plasmon resonance technique as described for illustration in Example 14.

Once isolated, at least a portion of the amino acid sequence of the adhesin can be ascertained using techniques well known to those of skill in the art, such as via the Edman degradation technique (see, e.g., Creighton, 1983, “Proteins: Structures and Molecular Principles”, W.H. Freeman & Co., N.Y., pp. 34-49) and/or mass spectrometry. The amino acid sequence obtained may be used as a guide for the generation of oligonucleotide mixtures that can be used to screen for a gene sequence encoding the adhesin. Screening may be accomplished, for example, by standard hybridization or PCR techniques. Techniques for the generation of oligonucleotide mixtures and the screening are well-known. (See, e.g., Ausubel, supra., and PCR Protocols: A Guide to Methods and Applications, 1990, Innis, M. et al., eds. Academic Press, Inc., New York).

Additionally, methods may be employed which result in the simultaneous identification of genes which encode the adhesin. These methods include, for example, probing expression libraries with a labelled antigen-binding molecule that is immuno-interactive specifically with an adherovar of the invention. Alternatively, reference may be made to Renner et al. (U.S. Pat. No. 6,197,502) who describe a method for identifying nucleic acids encoding proteins with a predetermined property of interest, including a particular cellular localization, structure, enzymatic function, or affinity to other molecules. In a first step, a plurality of eukaryotic host cells is provided, wherein each host cell has an expression system comprising a different member, each member comprising a recombinant nucleic acid encoding an exogenous protein operatively linked to a control element (e.g., a transcriptional control element such as a promoter). In a second step, the eukaryotic host cells are cultured under conditions where the exogenous protein is expressed while expression of endogenous proteins of the eukaryotic host cell is suppressed. In this time window, the exogenous protein may optionally be labelled, or may be treated in a way that allows discrimination from the untreated exogenous proteins. Finally, the member or members of the expression system that encode the exogenous protein or proteins having the property of interest (sperm binding) are identified. Accordingly, one may select for cells expressing an exogenous protein based on the exogenous protein being a membrane protein with an extracellular domain. For example, one may express exogenous proteins in cells while expression of endogenous proteins is inhibited or while using an expression system the operation of which inhibits the expression of endogenous proteins. Using this setup, one may, for example, treat the cells with proteases after inhibition of expression of endogenous proteins has set in, but while expression of exogenous protein is still continuing. After the cell surface has been deprived of extracellular proteinaceous protrusions, i.e., cellular membrane receptors and so on, the expression of the exogenous protein may replenish such protrusions. However, such replenishment will only occur if the exogenous protein expressed in a particular cell is a membrane receptor molecule. Therefore, at this step, only cells containing an expression vector or system that encodes a membrane associated molecule will have extracellular proteinaceous protrusions. Such protrusions may be used to bind the cell to any structure that binds proteins, regardless of whether such binding occurs specifically or non-specifically with regard to the structure of the protein that is bound. Cells bound to such a protein binding structure, may then, for example be further analyzed as to the sequence of the nucleic acid encoding the exogenous protein. A variety of structures that bind proteins have been described and are well known to the skilled artisan. For example, nitrocellulose, PVPF or nylon, filters may be used in this embodiment as a protein binding structure.

4. Preparation of Recombinant Adhesin Polypeptides

Once an adhesin-encoding gene is isolated and cloned, a recombinant adhesin protein or a fragment thereof may be prepared by expressing the coding sequence of the adhesin gene or a biologically active fragment thereof in an appropriate expression system. A recombinant adhesin protein may be prepared by any suitable procedure known to those of skill in the art. For example, the recombinant adhesin polypeptide may be prepared by a procedure including the steps of:

(a) preparing a recombinant polynucleotide comprising a nucleotide sequence encoding an adhesin protein, or a biologically active fragment thereof, or a variant or derivative of these, which nucleotide sequence is operably linked to transcriptional and translational control elements;

(b) introducing the recombinant polynucleotide into a suitable host cell;

(c) culturing the host cell to express recombinant polypeptide from said recombinant polynucleotide; and

(d) isolating the recombinant polypeptide.

The recombinant polynucleotide is preferably in the form of an expression vector that may be a self-replicating extra-chromosomal vector such as a plasmid, or a vector that integrates into a host genome.

The transcriptional and translational control elements will generally be appropriate for the host cell used for expression. Numerous types of appropriate expression vectors and suitable regulatory sequences are known in the art for a variety of host cells. Typically, the transcriptional and translational control elements may include, but are not limited to, promoter sequences, leader or signal sequences, ribosomal binding sites, transcriptional start and stop sequences, translational start and termination sequences, and enhancer or activator sequences. Constitutive or inducible promoters as known in the art are contemplated by the invention. The promoters may be either naturally occurring promoters, or hybrid promoters that combine elements of more than one promoter.

In a preferred embodiment, the expression vector contains a selectable marker gene to allow the selection of transformed host cells. Selection genes are well known in the art and will vary with the host cell used.

The expression vector may also include a fusion partner (typically provided by the expression vector) so that the recombinant polypeptide of the invention is expressed as a fusion polypeptide with said fusion partner. The main advantage of fusion partners is that they assist identification and/or purification of said fusion polypeptide. In order to express said fusion polypeptide, it is necessary to ligate a polynucleotide according to the invention into the expression vector so that the translational reading frames of the fusion partner and the polynucleotide coincide. Well-known examples of fusion partners include, but are not limited to, glutathione-S-transferase (GST), Fc potion of human IgG, maltose binding protein (MBP) and hexahistidine (HIS₆), which are particularly useful for isolation of the fusion polypeptide by affinity chromatography. For the purposes of fusion polypeptide purification by affinity chromatography, relevant matrices for affinity chromatography are glutathione-, amylose-, and nickel- or cobalt-conjugated resins respectively. Many such matrices are available in “kit” form, such as the QIAexpress™ system (Qiagen) useful with (HIS₆) fusion partners and the Pharmacia GST purification system. In a preferred embodiment, the recombinant polynucleotide is expressed in the commercial vector pFLAG as described more fully hereinafter. Another fusion partner well known in the art is green fluorescent protein (GFP). This fusion partner serves as a fluorescent “tag” which allows the fusion polypeptide of the invention to be identified by fluorescence microscopy or by flow cytometry. The GFP tag is useful when assessing subcellular localization of the fusion polypeptide of the invention, or for isolating cells which express the fusion polypeptide of the invention. Flow cytometric methods such as fluorescence activated cell sorting (FACS) are particularly useful in this latter application. Preferably, the fusion partners also have protease cleavage sites, such as for Factor Xₐ or Thrombin, which allow the relevant protease to partially digest the fusion polypeptide of the invention and thereby liberate the recombinant polypeptide of the invention therefrom. The liberated polypeptide can then be isolated from the fusion partner by subsequent chromatographic separation. Fusion partners according to the invention also include within their scope “epitope tags”, which are usually short peptide sequences for which a specific antibody is available. Well known examples of epitope tags for which specific monoclonal antibodies are readily available include c-Myc, influenza virus, haemagglutinin and FLAG tags.

The step of introducing into the host cell the recombinant polynucleotide may be effected by any suitable method including transfection, and transformation, the choice of which will be dependent on the host cell employed. Such methods are well known to those of skill in the art.

Recombinant polypeptides of the invention may be produced by culturing a host cell transformed with an expression vector containing nucleic acid encoding a polypeptide, biologically active fragment, variant or derivative according to the invention. The conditions appropriate for protein expression will vary with the choice of expression vector and the host cell. This is easily ascertained by one skilled in the art through routine experimentation. Suitable host cells for expression may be prokaryotic or eukaryotic. One preferred host cell for expression of a polypeptide according to the invention is a bacterium. The bacterium used may be Escherichia coli. Alternatively, the host cell may be an insect cell such as, for example, SF9 cells that may be utilised with a baculovirus expression system.

The recombinant protein may be conveniently prepared by a person skilled in the art using standard protocols as for example described in Sambrook, et al., MOLECULAR CLONING. A LABORATORY MANUAL (Cold Spring Harbor Press, 1989), in particular Sections 16 and 17; Ausubel et al., CURRENT PROTOCOLS IN MOLECULAR BIOLOGY (John Wiley & Sons, Inc. 1994-1998), in particular Chapters 10 and 16; and Coligan et al., CURRENT PROTOCOLS IN PROTEIN SCIENCE (John Wiley & Sons, Inc. 1995-1997), in particular Chapters 1, 5 and 6.

Alternatively, the polypeptide, fragments, variants or derivatives of the invention may be synthesized using solution synthesis or solid phase synthesis as described, for example, in Chapter 9 of Atherton and Shephard (supra) and in Roberge et al (1995, Science 269: 202).

The isolation of the adhesin sequence will enable one of ordinary skill in the art to identify and isolate nucleic acids which encode homologous adhesin proteins in other organisms of the invention, especially ureaplasmas. One of ordinary skill in the art may screen preparations of genomic or cDNA obtained from other organisms or from bacterial or other genomic or cDNA libraries using probes or PCR primers to identify homologous or variant sequences by standard hybridization screening or PCR techniques. For example, suitable polynucleotide sequence variants may be prepared according to the following procedure:
creating primers which are optionally degenerate wherein each comprises a portion of a reference polynucleotide encoding a reference adhesin protein, or a biologically active fragment thereof;
obtaining a nucleic acid extract from an adherovar, which is preferably a ureaplasma; and
using said primers to amplify, via nucleic acid amplification techniques, at least one amplification product from said nucleic acid extract, wherein said amplification product corresponds to a polynucleotide variant.

Suitable nucleic acid amplification techniques are well known to the skilled artisan, and include PCR as for example described by Ausubel et al. (supra); strand displacement amplification (SDA) as for example described in U.S. Pat. No. 5,422,252; rolling circle replication (RCR) as for example described by Liu et al., (1996, J. Am. Chem. Soc. 118:1587-1594; and International application WO 92/01813) and by Lizardi et al., (International Application WO 97/19193); nucleic acid sequence-based amplification (NASBA) as for example described by Sooknanan et al., (1994); and Q-β replicase amplification as for example described by Tyagi et al., (1996, Proc. Natl. Acad. Sci. USA 93: 5395-5400).

Alternatively, polynucleotide variants that are substantially complementary to a reference adhesin-encoding polynucleotide are identified by blotting techniques that include a step whereby nucleic acids are immobilized on a matrix (preferably a synthetic membrane such as nitrocellulose), followed by a hybridization step, and a detection step. Southern blotting is used to identify a complementary DNA sequence; northern blotting is used to identify a complementary RNA sequence. Dot blotting and slot blotting can be used to identify complementary DNA/DNA, DNA/RNA or RNA/RNA polynucleotide sequences. Such techniques are well known by those skilled in the art, and have been described in Ausubel et al. (1994-1998, supra) at pages 2.9.1 through 2.9.20.

Polynucleotides encoding at least a portion of an adhesin protein of an adherovar typified by the embodiments described herein are advantageous as nucleic acid probes for the identification of unique sequences found in other adherovars, especially in different strains or serotypes of ureaplasma and for the identification of adherovar infection or colonization, especially ureaplasma infection or colonization, as for example described infra in Section 5.1. Such polynucleotides are also useful for identifying and isolating modulatory agents that can modulate the level and/or functional activity of adhesin protein according to the invention, as for example described in Sections 6 and 7.

The products encoded by the above polynucleotide are also useful for identifying and isolating modulatory agents that can modulate the level and/or functional activity of adhesin protein according to the invention, as for example described in Sections 6 and 7. Additionally, the products encoded by the above polynucleotides are useful as antigens for the production of adherovar specific antigen-binding molecules as for example described infra in Section 5.2 and for vaccination against diseases, conditions or activities associated with adherovars, as for example described in Section 8. The adhesin polypeptides, biologically active fragments, variants and derivatives encoded by the aforementioned polynucleotides, and peptides containing sequences corresponding to portions of the adhesin that are conserved between various adherovar isolates that produce the adhesin, are useful in diagnosis of and immunisation against diseases or conditions associated with any organism that produces the adhesin protein, or an adhesin protein of similar binding specificity.

5. Methods of Detecting an Adhesin of the Invention

The present invention is predicated in part on the discovery that sperm infected or colonized with adherovars as disclosed herein, is more likely to impact adversely on male fertility and on pregnancy outcomes, including ART pregnancy outcomes, than uninfected/uncolonized sperm, or sperm infected or colonized with a weakly sperm adherent organism. Thus, the invention features a method for diagnosis or the prognostic assessment of (a) a higher risk of male infertility, (b) a higher risk of an adverse pregnancy outcome or (c) a higher risk of an adverse assisted reproductive technology (ART) outcome, comprising detecting an adherovar in a biological sample obtained from the patient or from the patient's sperm donor or detecting an adhesin of an adherovar, or a genetic sequence encoding the adhesin. The patient, in one embodiment, is a male whose sperm is the subject of donation. Thus, a suitable biological sample, which can be used for detection of the adherovar includes, but is not limited to, blood, serum, plasma, saliva, urine, sweat, ascitic fluid, peritoneal fluid, synovial fluid, and cerebrospinal fluid, seminiferous tubule fluid (e.g., in vasectomized men), semen, spermatozoa and seminiferous tubules. In an alternate embodiment, the patient is a female who is the subject of a fertilization or a donation of an oocyte. A biological sample obtained from the female, which can be used for detection of an adherovar includes, but is not restricted to blood, serum, plasma, saliva, urine, sweat, ascitic fluid, peritoneal fluid, synovial fluid, and cerebrospinal fluid, amniotic fluid, vaginal secretions, endocervical secretions, respiratory secretions, endometrial lining, fallopian tube washings, follicular fluid, placental tissue, ovarian tissue, fallopian tube tissue, oocyte and embryo tissue.

5.1 Genetic Diagnosis

One embodiment of the instant invention comprises a method for detecting a genetic sequence encoding a surface associated adhesin polypeptide of an adherovar. Nucleic acid used in polynucleotide-based assays can be isolated from cells contained in the biological sample (ureaplasma cells), according to standard methodologies (Sambrook, et al., “Molecular Cloning. A Laboratory Manual”, Cold Spring Harbor Press, 1989; Ausubel et al., “Current Protocols in Molecular Biology”, John Wiley & Sons Inc, 1994-1998). The nucleic acid may be genomic DNA or fractionated or whole cell RNA. Where RNA is used, it may be desired to convert the RNA to a complementary DNA. In one embodiment, the RNA is whole cell RNA; in another, it is poly-A RNA. In one embodiment, the nucleic acid is amplified by a nucleic acid amplification technique. Suitable nucleic acid amplification techniques are well known to the skilled addressee, and include the polymerase chain reaction (PCR) as for example described in Ausubel et al. (supra) as well as modifications thereof including real-time PCR as for example described by Lee, et al. (1993, Nucl. Acids Res. 21: 3761-3766), Ghosh, et al. (1994, Nucl. Acids Res. 22: 3155-3159), Han, et al. (International application WO 96/21144) and Nadeau, et al. (U.S. Pat. No. 5,846,726); strand displacement amplification (SDA) as for example described in U.S. Pat. No. 5,422,252; rolling circle replication (RCR) as for example described in Liu et al., (1996) and International application WO 92/01813) and Lizardi et al., (International Application WO 97/19193); nucleic acid sequence-based amplification (NASBA) as for example described by Sooknanan et al., (1994, Biotechniques 17:1077-1080); and Q-β replicase amplification as for example described by Tyagi et al., (1996, Proc. Natl. Acad. Sci. USA 93: 5395-5400).

Depending on the format, the specific nucleic acid of interest is identified in the sample directly using amplification or with a second, known nucleic acid following amplification. Next, the identified product is detected. In certain applications, the detection may be performed by visual means (e.g., ethidium bromide staining of a gel). Alternatively, the detection may involve indirect identification of the product via chemiluminescence, radioactive scintigraphy of radiolabel or fluorescent label or even via a system using electrical or thermal impulse signals (Affymax Technology; Bellus, 1994, J Macromol. Sci. Pure, Appl. Chem., A31(1): 1355-1376).

Following detection, one may compare the results seen in a given patient with a control reaction or a statistically significant reference group of normal patients. In this way, it is possible to correlate the presence or amount of an adhesin-encoding polynucleotide detected with the propensity (a) for male infertility, (b) for an adverse pregnancy outcome or (c) for an adverse assisted reproductive technology (ART) outcome.

5.1.1 Primers and Probes

Primers may be provided in double-stranded or single-stranded form, although the single-stranded form is preferred. Probes, while perhaps capable of priming, are designed to bind to a target DNA or RNA and need not be used in an amplification process. In preferred embodiments, the probes or primers are labelled with radioactive species ³²P, ¹⁴C, ³⁵S, ³H, or other label), with a fluorophore (rhodamine, fluorescein) or a chemillumiscent label (luciferase).

5.1.2 Template Dependent Amplification Methods

A number of template dependent processes are available to amplify the marker sequences present in a given template sample. An exemplary nucleic acid amplification technique is the polymerase chain reaction (referred to as PCR) which is described in detail in U.S. Pat. Nos. 4,683,195, 4,683,202 and 4,800,159, Ausubel et al. (supra), and in Innis et al., (“PCR Protocols”, Academic Press, Inc., San Diego Calif., 1990).

Briefly, in PCR, two primer sequences are prepared that are complementary to regions on opposite complementary strands of the marker sequence. An excess of deoxynucleoside triphosphates are added to a reaction mixture along with a DNA polymerase, e.g., Taq polymerase. If the marker sequence is present in a sample, the primers will bind to the marker and the polymerase will cause the primers to be extended along the marker sequence by adding on nucleotides. By raising and lowering the temperature of the reaction mixture, the extended primers will dissociate from the marker to form reaction products, excess primers will bind to the marker and to the reaction products and the process is repeated.

A reverse transcriptase PCR amplification procedure may be performed in order to quantify the amount of mRNA amplified. Methods of reverse transcribing RNA into cDNA are well known and described in Sambrook et al., 1989. Alternative methods for reverse transcription utilise thermostable, RNA-dependent DNA polymerases. These methods are described in WO 90/07641. Polymerase chain reaction methodologies are well known in the art.

Another method for amplification is the ligase chain reaction (“LCR”), disclosed in EPO No. 320 308. In LCR, two complementary probe pairs are prepared, and in the presence of the target sequence, each pair will bind to opposite complementary strands of the target such that they abut. In the presence of a ligase, the two probe pairs will link to form a single unit. By temperature cycling, as in PCR, bound ligated units dissociate from the target and then serve as “target sequences” for ligation of excess probe pairs. U.S. Pat. No. 4,883,750 describes a method similar to LCR for binding probe pairs to a target sequence.

Qβ Replicase, described in PCT Application No. PCT/US87/00880, may also be used as still another amplification method in the present invention. In this method, a replicative sequence of RNA that has a region complementary to that of a target is added to a sample in the presence of an RNA polymerase. The polymerase will copy the replicative sequence that can then be detected.

An isothermal amplification method, in which restriction endonucleases and ligases are used to achieve the amplification of target molecules that contain nucleotide 5′α-thio-triphosphates in one strand of a restriction site may also be useful in the amplification of nucleic acids in the present invention, Walker et al., (1992, Proc. Natl. Acad. Sci. U.S.A 89: 392-396).

Strand Displacement Amplification (SDA) is another method of carrying out isothermal amplification of nucleic acids which involves multiple rounds of strand displacement and synthesis, i.e., nick translation. A similar method, called Repair Chain Reaction (RCR), involves annealing several probes throughout a region targeted for amplification, followed by a repair reaction in which only two of the four bases are present. The other two bases can be added as biotinylated derivatives for easy detection. A similar approach is used in SDA. Target specific sequences can also be detected using a cyclic probe reaction (CPR). In CPR, a probe having 3′ and 5′ sequences of non-specific DNA and a middle sequence of specific RNA is hybridized to DNA that is present in a sample. Upon hybridization, the reaction is treated with RNase H, and the products of the probe identified as distinctive products that are released after digestion. The original template is annealed to another cycling probe and the reaction is repeated.

Still another amplification methods described in GB Application No. 2 202 328, and in PCT Application No. PCT/US89/01025, may be used in accordance with the present invention. In the former application, “modified” primers are used in a PCR-like, template- and enzyme-dependent synthesis. The primers may be modified by labelling with a capture moiety (e.g., biotin) and/or a detector moiety (e.g., enzyme). In the latter application, an excess of labelled probes are added to a sample. In the presence of the target sequence, the probe binds and is cleaved catalytically. After cleavage, the target sequence is released intact to be bound by excess probe. Cleavage of the labelled probe signals the presence of the target sequence.

Other nucleic acid amplification procedures include transcription-based amplification systems (TAS), including nucleic acid sequence based amplification (NASBA) and 3SR (Kwoh et al., 1989, Proc. Natl. Acad. Sci. U.S.A., 86: 1173; Gingeras et al., PCT Application WO 88/10315). In NASBA, the nucleic acids can be prepared for amplification by standard phenol/chloroform extraction, heat denaturation of a clinical sample, treatment with lysis buffer and minispin columns for isolation of DNA and RNA or guanidinium chloride extraction of RNA. These amplification techniques involve annealing a primer which has target specific sequences. Following polymerzation, DNA/RNA hybrids are digested with RNase H while double stranded DNA molecules are heat denatured again. In either case the single stranded DNA is made fully double stranded by addition of second target specific primer, followed by polymerzation. The double-stranded DNA molecules are then multiply transcribed by an RNA polymerase such as T7 or SP6. In an isothermal cyclic reaction, the RNAs are reverse transcribed into single stranded DNA, which is then converted to double stranded DNA, and then transcribed once again with an RNA polymerase such as T7 or SP6. The resulting products, whether truncated or complete, indicate target specific sequences.

Davey et al., EPO No. 329 822 disclose a nucleic acid amplification process involving cyclically synthesizing single-stranded RNA (“ssRNA”), ssDNA, and double-stranded DNA (dsDNA), which may be used in accordance with the present invention. The ssRNA is a template for a first primer oligonucleotide, which is elongated by reverse transcriptase (RNA-dependent DNA polymerase). The RNA is then removed from the resulting DNA:RNA duplex by the action of ribonuclease H(RNase H, an RNase specific for RNA in duplex with either DNA or RNA). The resultant ssDNA is a template for a second primer, which also includes the sequences of an RNA polymerase promoter (exemplified by T7 RNA polymerase) 5′ to its homology to the template. This primer is then extended by DNA polymerase (exemplified by the large “Klenow” fragment of E. coli DNA polymerase I), resulting in a double-stranded DNA (“dsDNA”) molecule, having a sequence identical to that of the original RNA between the primers and having additionally, at one end, a promoter sequence. This promoter sequence can be used by the appropriate RNA polymerase to make many RNA copies of the DNA. These copies can then re-enter the cycle leading to very swift amplification. With proper choice of enzymes, this amplification can be done isothermally without addition of enzymes at each cycle. Because of the cyclical nature of this process, the starting sequence can be chosen to be in the form of either DNA or RNA.

Miller et al. in PCT Application WO 89/06700 disclose a nucleic acid sequence amplification scheme based on the hybridization of a promoter/primer sequence to a target single-stranded DNA (“ssDNA”) followed by transcription of many RNA copies of the sequence. This scheme is not cyclic, i.e., new templates are not produced from the resultant RNA transcripts. Other amplification methods include “RACE” and “one-sided PCR” (Frohman, M. A., In: “PCR Protocols: A Guide to Methods and Applications”, Academic Press, N.Y., 1990; Ohara et al., 1989, Proc. Natl. Acad. Sci. U.S.A., 86: 5673-567).

Methods based on ligation of two (or more) oligonucleotides in the presence of nucleic acid having the sequence of the resulting “di-oligonucleotide”, thereby amplifying the di-oligonucleotide, may also be used in the amplification step of the present invention. Wu et al., (1989, Genomics 4: 560).

5.1.3 Southern/Northern Blotting

Blotting techniques are well known to those of skill in the art. Southern blotting involves the use of DNA as a target, whereas Northern blotting involves the use of RNA as a target. Each provide different types of information, although cDNA blotting is analogous, in many aspects, to blotting or RNA species.

Briefly, a probe is used to target a DNA or RNA species that has been immobilized on a suitable matrix, often a filter of nitrocellulose. The different species should be spatially separated to facilitate analysis. This often is accomplished by gel electrophoresis of nucleic acid species followed by “blotting” on to the filter.

Subsequently, the blotted target is incubated with a probe (usually labelled) under conditions that promote denaturation and rehybridization. Because the probe is designed to base pair with the target, the probe will bind a portion of the target sequence under renaturing conditions. Unbound probe is then removed, and detection is accomplished as described above.

Alternatively, an in situ hybridization technique may be employed to detect a target nucleic acid sequence at the cellular or tissue level. An exemplary technique of this kind includes fluorescent in situ hybridization (FISH) as, for example, described by Verma, et al. (1988, Human Chromosomes: A Manual of Basic Techniques, Pergamon Press, New York, N.Y.).

5.1.4 Detection Methods

Products may be visualised in order to confirm amplification of the marker sequences. One typical visualisation method involves staining of a gel with ethidium bromide and visualisation under UV light. Alternatively, if the amplification products are integrally labelled with radio- or fluorometrically-labelled nucleotides, the amplification products can then be exposed to x-ray film or visualised under the appropriate stimulating spectra, following separation.

In one embodiment, visualisation is achieved indirectly. Following separation of amplification products, a labelled nucleic acid probe is brought into contact with the amplified marker sequence. The probe preferably is conjugated to a chromophore but may be radiolabelled. In another embodiment, the probe is conjugated to a binding partner, such as an antibody or biotin, and the other member of the binding pair carries a detectable moiety or reporter molecule.

In one embodiment, detection is by a labelled probe. The techniques involved are well known to those of skill in the art and can be found in many standard texts on molecular protocols. See Sambrook et al., 1989. For example, chromophore or radiolabel probes or primers identify the target during or following amplification.

One example of the foregoing is described in U.S. Pat. No. 5,279,721, which discloses an apparatus and method for the automated electrophoresis and transfer of nucleic acids. The apparatus permits electrophoresis and blotting without external manipulation of the gel and is ideally suited to carrying out methods according to the present invention.

In addition, the amplification products described above may be subjected to sequence analysis to identify specific kinds of variations using standard sequence analysis techniques. Within certain methods, exhaustive analysis of genes is carried out by sequence analysis using primer sets designed for optimal sequencing (Pignon et al., 1994, Hum. Mutat. 3: 126-132). The present invention provides methods by which any or all of these types of analyses may be used. Once an adhesin-encoding polynucleotide is isolated and its sequence determined, oligonucleotide primers may be designed to permit the amplification of sequences throughout that polynucleotide that may then be analyzed by direct sequencing.

Once the adhesin genes from adherovars and from less adherent serovars have been sequenced and polymorphic base pair differences defined then these polymorphic sequences may be detected using DNA based technologies which are capable of detecting and optionally distinguishing polymorphisms.

5.1.5 Kit Components

All the essential materials and reagents required for detecting and sequencing adhesin-encoding polynucleotides and variants thereof may be assembled together in a kit. The kits may also optionally include appropriate reagents for detection of labels, positive and negative controls, washing solutions, dilution buffers and the like. For example, a nucleic acid-based detection kit may include (i) a polynucleotide according to the invention (which may be used as a positive control), (ii) an oligonucleotide primer according to the invention. Also included may be enzymes suitable for amplifying nucleic acids including various polymerases (Reverse Transcriptase, Taq, Sequenase™ DNA ligase etc. depending on the nucleic acid amplification technique employed), deoxynucleotides and buffers to provide the necessary reaction mixture for amplification. Such kits also generally will comprise, in suitable means, distinct containers for each individual reagent and enzyme as well as for each primer or probe.

5.1.6 Chip Technologies

Also contemplated by the present invention are chip-based DNA technologies such as those described by Hacia et al. (1996, Nature Genetics 14: 441-447) and Shoemaker et al. (1996, Nature Genetics 14: 450-456). Briefly, these techniques involve quantitative methods for analysing large numbers of genes rapidly and accurately. By tagging genes with oligonucleotides or using fixed probe arrays, one can employ chip technology to segregate target molecules as high density arrays and screen these molecules on the basis of hybridization. See also Pease et al. (1994, Proc. Natl. Acad. Sci. U.S.A. 91: 5022-5026); Fodor et al. (1991, Science 251: 767-773).

5.2 Protein-Based Diagnostics

5.2.1 Antigen-Binding Molecules

The invention also contemplates antigen-binding molecules that are specifically immuno-interactive with an adherovar of the invention and particularly with an adherovar adhesin of the invention, or with fragments thereof, or with variants or derivatives of these. For example, the antigen-binding molecules may comprise whole polyclonal antibodies. Such antibodies may be prepared, for example, by injecting a polypeptide, fragment, variant or derivative of the invention into a production species, which may include mice or rabbits, to obtain polyclonal antisera. Methods of producing polyclonal antibodies are well known to those skilled in the art. Exemplary protocols which may be used are described for example in Coligan et al., CURRENT PROTOCOLS IN IMMUNOLOGY, (John Wiley & Sons, Inc, 1991), and Ausubel et al., (1994-1998, supra), in particular Section III of Chapter 11.

In lieu of the polyclonal antisera obtained in the production species, monoclonal antibodies may be produced using the standard method as described, for example, by Köhler and Milstein (1975, Nature 256, 495497), or by more recent modifications thereof as described, for example, in Coligan et al., (1991, supra) by immortalising spleen or other antibody-producing cells derived from a production species which has been inoculated with one or more of the polypeptides, polypeptide fragments, variants or derivatives of the invention.

The invention also contemplates as antigen-binding molecules Fv, Fab, Fab′ and F(ab′)₂ immunoglobulin fragments or other synthetic antigen-binding molecules such as synthetic stabilised Fv fragments, dAbs, minibodies and the like, which can be produced using routine methods by practitioners in the art.

The antigen-binding molecules of the invention may be used for affinity chromatography in isolating a natural or recombinant polypeptide or biologically active fragment of the invention. For example reference may be made to immunoaffinity chromatographic procedures described in Chapter 9.5 of Coligan et al., (1995-1997, supra). The antigen-binding molecules can also be used to screen expression libraries for variant polypeptides of the invention as described herein. They can also be used to detect polypeptides, fragments, variants and derivatives of the invention as described hereinafter.

5.2.2 Immunodiagnostic Assays

The above antigen-binding molecules have utility in detecting adhesins of adherovars typified by the embodiments described herein, through techniques such as ELISAs and Western blotting. Illustrative assay strategies which can be used to detect a target polypeptide of the invention include, but are not limited to, immunoassays involving the binding of an antigen-binding molecule to the target polypeptide (e.g., an adhesin polypeptide) in the sample, and the detection of a complex comprising the antigen-binding molecule and the target polypeptide. Preferred immunoassays are those that can measure the level and/or functional activity of a target molecule of the invention. Typically, an antigen-binding molecule that is immuno-interactive with a target polypeptide of the invention is contacted with a biological sample suspected of containing said target polypeptide. The concentration of a complex comprising the antigen-binding molecule and the target polypeptide is measured and the measured complex concentration is then related to the concentration of target polypeptide in the sample.

Any suitable technique for determining formation of an antigen-binding molecule-target antigen complex may be used. For example, an antigen-binding molecule according to the invention, having a reporter molecule associated therewith may be utilised in immunoassays. Such immunoassays include, but are not limited to, radioimmunoassays (RIAs), enzyme-linked immunosorbent assays (ELISAs) and immunochromatographic techniques (ICTs), Western blotting which are well known those of skill in the art. For example, reference may be made to Coligan et al. (1994, supra) which discloses a variety of immunoassays that may be used in accordance with the present invention. Immunoassays may include competitive assays as understood in the art or as for example described infra. It will be understood that the present invention encompasses qualitative and quantitative immunoassays.

Suitable immunoassay techniques are described for example in U.S. Pat. Nos. 4,016,043, 4, 424,279 and 4,018,653. These include both single-site and two-site assays of the non-competitive types, as well as the traditional competitive binding assays. These assays also include direct binding of a labelled antigen-binding molecule to a target antigen.

Two site assays are particularly favoured for use in the present invention. A number of variations of these assays exist, all of which are intended to be encompassed by the present invention. Briefly, in a typical forward assay, an unlabelled antigen-binding molecule such as an unlabelled antibody is immobilized on a solid substrate and the sample to be tested brought into contact with the bound molecule. After a suitable period of incubation, for a period of time sufficient to allow formation of an antibody-antigen complex, another antigen-binding molecule, suitably a second antibody specific to the antigen, labelled with a reporter molecule capable of producing a detectable signal is then added and incubated, allowing time sufficient for the formation of another complex of antibody-antigen-labelled antibody. Any unreacted material is washed away and the presence of the antigen is determined by observation of a signal produced by the reporter molecule. The results may be either qualitative, by simple observation of the visible signal, or may be quantitated by comparing with a control sample containing known amounts of antigen. Variations on the forward assay include a simultaneous assay, in which both sample and labelled antibody are added simultaneously to the bound antibody. These techniques are well known to those skilled in the art, including minor variations as will be readily apparent. In accordance with the present invention, the sample is one that might contain an antigen including a tissue or fluid as described above.

In the typical forward assay, a first antibody having specificity for the antigen or antigenic parts thereof is either covalently or passively bound to a solid surface. The solid surface is typically glass or a polymer, the most commonly used polymers being cellulose, polyacrylamide, nylon, polystyrene, polyvinyl chloride or polypropylene. The solid supports may be in the form of tubes, beads, discs of microplates, or any other surface suitable for conducting an immunoassay. The binding processes are well known in the art and generally consist of cross-linking covalently binding or physically adsorbing, the polymer-antibody complex is washed in preparation for the test sample. An aliquot of the sample to be tested is then added to the solid phase complex and incubated for a period of time sufficient and under suitable conditions to allow binding of any antigen present to the antibody. Following the incubation period, the antigen-antibody complex is washed and dried and incubated with a second antibody specific for a portion of the antigen. The second antibody has generally a reporter molecule associated therewith that is used to indicate the binding of the second antibody to the antigen. The amount of labelled antibody that binds, as determined by the associated reporter molecule, is proportional to the amount of antigen bound to the immobilized first antibody.

An alternative method involves immobilizing the antigen in the biological sample and then exposing the immobilized antigen to specific antibody that may or may not be labelled with a reporter molecule. Depending on the amount of target and the strength of the reporter molecule signal, a bound antigen may be detectable by direct labelling with the antibody. Alternatively, a second labelled antibody, specific to the first antibody is exposed to the target-first antibody complex to form a target-first antibody-second antibody tertiary complex. The complex is detected by the signal emitted by the reporter molecule.

From the foregoing, it will be appreciated that the reporter molecule associated with the antigen-binding molecule may include the following: (a) direct attachment of the reporter molecule to the antigen-binding molecule; (b) indirect attachment of the reporter molecule to the antigen-binding molecule; i.e., attachment of the reporter molecule to another assay reagent which subsequently binds to the antigen-binding molecule; and (c) attachment to a subsequent reaction product of the antigen-binding molecule.

The reporter molecule may be selected from a group including a chromogen, a catalyst, an enzyme, a fluorochrome, a chemiluminescent molecule, a lanthanide ion such as Europium (Eu³⁴), a radioisotope and a direct visual label.

In the case of a direct visual label, use may be made of a colloidal metallic or non-metallic particle, a dye particle, an enzyme or a substrate, an organic polymer, a latex particle, a liposome, or other vesicle containing a signal producing substance and the like.

A large number of enzymes suitable for use as reporter molecules is disclosed in United States Patent Specifications U.S. Pat. No. 4,366,241, U.S. Pat. No. 4,843,000, and U.S. Pat. No. 4,849,338. Suitable enzymes useful in the present invention include alkaline phosphatase, horseradish peroxidase, luciferase, β-galactosidase, glucose oxidase, lysozyme, malate dehydrogenase and the like. The enzymes may be used alone or in combination with a second enzyme that is in solution.

Suitable fluorochromes include, but are not limited to, fluorescein isothiocyanate (FITC), tetramethylrhodamine isothiocyanate (TRITC), R-Phycoerythrin (RPE), Texas Red and Alex Fluor probes. Other exemplary fluorochromes include those discussed by Dower et al. (International Publication WO 93/06121). Reference also may be made to the fluorochromes described in U.S. Pat. No. 5,573,909 (Singer et al), U.S. Pat. No. 5,326,692 (Brinkley et al). Alternatively, reference may be made to the fluorochromes described in U.S. Pat. Nos. 5,227,487, 5,274,113, 5,405,975, 5,433,896, 5,442,045, 5,451,663, 5,453,517, 5,459,276, 5,516,864, 5,648,270 and 5,723,218.

In the case of an enzyme immunoassay, an enzyme is conjugated to the second antibody, generally by means of glutaraldehyde or periodates. As will be readily recognised, however, a wide variety of different conjugation techniques exist which are readily available to the skilled artisan. The substrates to be used with the specific enzymes are generally chosen for the production of, upon hydrolysis by the corresponding enzyme, a detectable colour change. Examples of suitable enzymes include those described supra. It is also possible to employ fluorogenic substrates, which yield a fluorescent product rather than the chromogenic substrates noted above. In all cases, the enzyme-labelled antibody is added to the first antibody-antigen complex. It is then allowed to bind, and excess reagent is washed away. A solution containing the appropriate substrate is then added to the complex of antibody-antigen-antibody. The substrate will react with the enzyme linked to the second antibody, giving a qualitative visual signal, which may be further quantitated, usually spectrophotometrically, to give an indication of the amount of antigen which was present in the sample.

Alternately, fluorescent compounds, such as fluorescein, rhodamine and the lanthanide, europium (EU), may be chemically coupled to antibodies without altering their binding capacity. When activated by illumination with light of a particular wavelength, the fluorochrome-labelled antibody adsorbs the light energy, inducing a state to excitability in the molecule, followed by emission of the light at a characteristic colour visually detectable with a light microscope. The fluorescent-labelled antibody is allowed to bind to the first antibody-antigen complex. After washing off the unbound reagent, the remaining tertiary complex is then exposed to light of an appropriate wavelength. The fluorescence observed indicates the presence of the antigen of interest. Immunofluorometric assays (IFMA) are well established in the art. However, other reporter molecules, such as radioisotope, chemiluminescent or bioluminescent molecules may also be employed.

It will be well understood that other means of testing target polypeptide (e.g., an adhesin of the invention) levels are available, including, for instance, those involving testing for an altered level of adhesin binding activity to a spermatozoon or to a carbohydrate (e.g., glycolipid), or Western blot analysis of adhesin protein levels on ureaplasma or on spermatozoa, using an anti-adhesin antigen-binding molecule, or assaying the amount of antigen-binding molecule or other adhesin-binding partner which is not bound to a sample, and subtracting from the total amount of antigen-binding molecule or binding partner added.

6. Identification of Target Molecule Modulators

The invention also features a method of screening for an agent that modulates the expression of a gene or the level and/or functional activity of an expression product of that gene, wherein the gene is selected from an adhesin-encoding gene or a gene relating to the same regulatory or biosynthetic pathway as the adhesin-encoding gene. The method comprises contacting a preparation comprising (i) a polypeptide comprising an amino acid sequence corresponding to at least a biologically active fragment of said adhesin, or to a variant or derivative thereof; or (ii) a polynucleotide comprising at least a portion of a genetic sequence that regulates the expression of said polypeptide, which is operably linked to a reporter gene, with a test agent and detecting a change in the level and/or functional activity of said polypeptide, or an expression product of said reporter gene, relative to a normal or reference level and/or functional activity in the absence of said test agent.

Any suitable assay for detecting, measuring or otherwise determining modulation of adherence of said adhesin for a binding partner (e.g., a carbohydrate or sperm) is contemplated by the present invention. Assays of a suitable nature are known to persons of skill in the art. It will be understood, in this regard, that the present invention is not limited to the use or practice of any one particular assay for determining affinity of protein—protein interactions or strength of adherence between binding partners.

Modulatory compounds contemplated by the present invention includes agonists and antagonists of adhesin gene expression. Antagonists of adhesin gene expression include antisense molecules, ribozymes and co-suppression molecules. Agonists include molecules which increase promoter activity or interfere with negative mechanisms. Agonists of an adhesin according to the invention include molecules which overcome any negative regulatory mechanism. Antagonists of adhesin polypeptides include antibodies and inhibitor peptide or other fragments—e.g. glycolipid mimics (see for example U.S. Pat. No. 6,103,883).

Candidate agents encompass numerous chemical classes, though typically they are organic molecules, preferably small organic compounds having a molecular weight of more than 50 and less than about 2,500 Dalton. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including, but not limited to: peptides, saccharides, fatty acids, steroids, purines, pyrimidines, derivatives, structural analogues or combinations thereof.

Small (non-peptide) molecule modulators of an adhesin of the invention are particularly preferred. In this regard, small molecules are particularly preferred because such molecules are more readily absorbed after oral administration, have fewer potential antigenic determinants, and/or are more likely to cross the cell membrane than larger, protein-based pharmaceuticals. Small organic molecules may also have the ability to gain entry into an appropriate cell and affect the expression of a gene (e.g., by interacting with the regulatory region or transcription factors involved in gene expression); or affect the activity of a gene by inhibiting or enhancing the binding of accessory molecules.

Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogues. Screening may also be directed to known pharmacologically active compounds and chemical analogues thereof.

The present invention also contemplates the screening of larger molecules (>2,500 Dalton) for adhesin-modulating activity, which could be used for example in directly treating sperm ex vivo. Such modulators may be selected from polymeric molecules such as polynucleotides (e.g., antisense molecules or ribozymes that are specific for a gene encoding an adherovar adhesin) and polypeptides (e.g., antigen-binding molecules that are immuno-interactive with an adherovar adhesin or specific binding partners of an adherovar adhesin) or other polymers such as carbohydrates, glycolipids, glycoproteins etc as known in the art.

Screening for modulatory agents according to the invention can be achieved by any suitable method. For example, the method may include contacting a cell comprising a polynucleotide corresponding to an adhesin gene or to a gene belonging to the same regulatory or biosynthetic pathway as the adhesin gene, with an agent suspected of having said modulatory activity and screening for the modulation of the level and/or functional activity of a protein encoded by said polynucleotide, or the modulation of the level of an expression product encoded by the polynucleotide, or the modulation of the activity or expression of a downstream cellular target of said protein or said expression product. Detecting such modulation can be achieved utilising techniques including, but not restricted to, ELISA, cell-based ELISA, filter-binding ELISA, inhibition ELISA, Western blots, immunoprecipitation, slot or dot blot assays, immunostaining, RIA, scintillation proximity assays, fluorescent immunoassays using antigen-binding molecule conjugates or antigen conjugates of fluorescent substances such as fluorescein or rhodamine, Ouchterlony double diffusion analysis, immunoassays employing an avidin-biotin or a streptavidin-biotin detection system, and nucleic acid detection assays including reverse transcriptase PCR, and assays that interrogate specific binding partner interactions (e.g., using the BIAcore™ system). It will be understood that a polynucleotide from which a target molecule of interest is regulated or expressed may be naturally occurring in the cell which is the subject of testing or it may have been introduced into the host cell for the purpose of testing. Further, the naturally-occurring or introduced sequence may be constitutively expressed—thereby providing a model useful in screening for agents which down-regulate expression of an encoded product of the sequence wherein said down regulation can be at the nucleic acid or expression product level—or may require activation—thereby providing a model useful in screening for agents that up-regulate expression of an encoded product of the sequence. Further, to the extent that a polynucleotide is introduced into a cell, that polynucleotide may comprise the entire coding sequence which codes for a target protein or it may comprise a portion of that coding sequence (e.g. a domain such as a protein-binding domain or carbohydrate-binding domain) or a portion that regulates expression of a product encoded by the polynucleotide (e.g., a promoter). For example, the promoter that is naturally associated with the polynucleotide may be introduced into the cell that is the subject of testing. In this regard, where only the promoter is utilised, detecting modulation of the promoter activity can be achieved, for example, by operably linking the promoter to a suitable reporter polynucleotide including, but not restricted to, green fluorescent protein (GFP), luciferase, β-galactosidase and catecholamine acetyl transferase (CAT). Modulation of expression may be determined by measuring the activity associated with the reporter polynucleotide.

In another example, the subject of detection could be a downstream regulatory target of the target molecule, rather than target molecule itself or the reporter molecule operably linked to a promoter of a gene encoding a product the expression of which is regulated by the target protein.

These methods provide a mechanism for performing high throughput screening of putative modulatory agents such as proteinaceous or non-proteinaceous agents comprising synthetic, combinatorial, chemical and natural libraries. These methods will also facilitate the detection of agents which bind either the polynucleotide encoding the target molecule or which modulate the expression of an upstream molecule, which subsequently modulates the expression of the polynucleotide encoding the target molecule. Accordingly, these methods provide a mechanism of detecting agents that either directly or indirectly modulate the expression and/or activity of a target molecule according to the invention.

In a series of preferred embodiments, the present invention provides assays for identifying small molecules or other compounds (i.e., modulatory agents) which are capable of inducing or inhibiting the level and/or or functional activity of target molecules according to the invention. The assays may be performed in vitro using non-transformed cells (e.g., ureaplasma cells), immortalised cells, or recombinant cells. In addition, the assays may detect the presence of increased or decreased expression of genes or production of proteins on the basis of increased or decreased mRNA expression (using, for example, the nucleic acid probes disclosed herein), increased or decreased levels of protein products (using, for example, the antigen binding molecules disclosed herein), or increased or decreased levels of expression of a reporter gene (e.g., GFP, β-galactosidase or luciferase) operatively linked to a target molecule-related gene regulatory region in a recombinant construct.

Thus, for example, one may culture cells which produce a particular target molecule and add to the culture medium one or more test compounds. After allowing a sufficient period of time (e.g., 6-72 hours) for the compound to induce or inhibit the level and/or functional activity of the target molecule, any change in said level from an established baseline may be detected using any of the techniques described above and well known in the art. Using the nucleic acid probes and/or antigen-binding molecules prepared for example according to protocols described herein, detection of changes in the level and or functional activity of a target molecule, and thus identification of the compound as agonist or antagonist of the target molecule, requires only routine experimentation.

In particularly preferred embodiments, a recombinant assay is employed in which a reporter gene encoding, for example, GFP, β-galactosidase or luciferase is operably linked to the 5′ regulatory regions of a target molecule related gene. Such regulatory regions may be easily isolated and cloned by one of ordinary skill in the art in light of the present disclosure. The reporter gene and regulatory regions are joined in-frame (or in each of the three possible reading frames) so that transcription and translation of the reporter gene may proceed under the control of the regulatory elements of the target molecule related gene. The recombinant construct may then be introduced into any appropriate cell type. The transformed cells may be grown in culture and, after establishing the baseline level of expression of the reporter gene, test compounds may be added to the medium. The ease of detection of the expression of the reporter gene provides for a rapid, high throughput assay for the identification of agonists or antagonists of the target molecules of the invention.

Compounds identified by this method will have potential utility in modifying the expression of target molecule related genes in vivo. These compounds may be further tested in the animal models to identify those compounds having the most potent in vivo effects. In addition, as described above with respect to small molecules having target polypeptide binding activity, these molecules may serve as “lead compounds” for the further development of pharmaceuticals by, for example, subjecting the compounds to sequential modifications, molecular modelling, and other routine procedures employed in rational drug design.

In another embodiment, a method of identifying agents that inhibit the adhesion of an adhesin of the invention is provided in which a purified preparation of adhesin protein is incubated in the presence and absence of a candidate agent, and the affinity of the adhesin for a spermatozoon or for a carbohydrate binding partner is measured by a suitable assay. For example, an adhesin inhibitor can be identified by measuring the ability of a candidate agent to decrease the level of the adhesin on the surface of a cell (e.g., a ureaplasma cell). In this method, a cell that is capable of expressing an adhesin-encoding gene is exposed to, or cultured in the presence and absence of, the candidate agent and the binding of the cell to a spermatozoon or to a carbohydrate binding partner is detected. An agent tests positive if it inhibits this binding.

In yet another embodiment, random peptide libraries consisting of all possible combinations of amino acids attached to a solid phase support may be used to identify peptides that are able to bind to a target molecule or to a functional domain thereof. Identification of molecules that are able to bind to a target molecule may be accomplished by screening a peptide library with a recombinant soluble target molecule. The target molecule may be purified, recombinantly expressed or synthesized by any suitable technique. Such molecules may be conveniently prepared by a person skilled in the art using standard protocols as for example described in Sambrook, et al., MOLECULAR CLONING. A LABORATORY MANUAL (Cold Spring Harbor Press, 1989), in particular Sections 16 and 17; Ausubel et al., CURRENT PROTOCOLS IN MOLECULAR BIOLOGY (John Wiley & Sons, Inc. 1994-1998), in particular Chapters 10 and 16; and Coligan et al., CURRENT PROTOCOLS IN PROTEIN SCIENCE (John Wiley & Sons, Inc. 1995-1997), in particular Chapters 1, 5 and 6. Alternatively, a target polypeptide according to the invention may be synthesized using solution synthesis or solid phase synthesis as described, for example, in Chapter 9 of Atherton and Shephard (supra) and in Roberge et al (1995, Science 269: 202).

To identify and isolate the peptide/solid phase support that interacts and forms a complex with a target molecule, preferably a target polypeptide, it may be necessary to label or “tag” the target polypeptide. The target polypeptide may be conjugated to any suitable reporter molecule, including enzymes such as alkaline phosphatase and horseradish peroxidase and fluorescent reporter molecules such as fluorescein isothyiocynate (FITC), phycoerythrin (PE) and rhodamine. Conjugation of any given reporter molecule, with target polypeptide, may be performed using techniques that are routine in the art. Alternatively, target polypeptide expression vectors may be engineered to express a chimeric target polypeptide containing an epitope for which a commercially available antigen-binding molecule exists. The epitope specific antigen-binding molecule may be tagged using methods well known in the art including labelling with enzymes, fluorescent dyes or coloured or magnetic beads.

For example, the “tagged” target polypeptide conjugate is incubated with the random peptide library for 30 minutes to one hour at 22° C. to allow complex formation between target polypeptide and peptide species within the library. The library is then washed to remove any unbound target polypeptide. If the target polypeptide has been conjugated to alkaline phosphatase or horseradish peroxidase the whole library is poured into a petri dish containing a substrate for either alkaline phosphatase or peroxidase, for example, 5-bromo-4-chloro-3-indoyl phosphate (BCIP) or 3,3′,4,4″-diamnobenzidine (DAB), respectively. After incubating for several minutes, the peptide/solid phase-target polypeptide complex changes colour, and can be easily identified and isolated physically under a dissecting microscope with a micromanipulator. If a fluorescently tagged target polypeptide has been used, complexes may be isolated by fluorescent activated sorting. If a chimeric target polypeptide having a heterologous epitope has been used, detection of the peptide/target polypeptide complex may be accomplished by using a labelled epitope specific antigen-binding molecule. Once isolated, the identity of the peptide attached to the solid phase support may be determined by peptide sequencing.

7. Method of Modulating the Level and/or Functional Activity of an Adhesin

The invention, therefore, provides a method for modulating adhesion of an adherovar to sperm, comprising contacting said adherovar with an agent for a time and under conditions sufficient to modulate the level and/or functional activity of an adhesin polypeptide as broadly described above. In a preferred embodiment, the agent decreases the level and/or functional activity of the adhesin protein. In such a case, the agent is suitably used to reduce, repress or otherwise inhibit adherence of an adherovar to sperm. Suitable adhesin inhibitors may be identified or produced by methods for example disclosed in Section 6.

For example, a suitable adhesin inhibitor may comprise oligoribonucleotide sequences, that include anti-sense RNA and DNA molecules and ribozymes that function to inhibit the translation of adhesin protein-encoding mRNA. Anti-sense RNA and DNA molecules act to directly block the translation of mRNA by binding to targeted mRNA and preventing protein translation. In regard to antisense DNA, oligodeoxyribonucleotides derived from the translation initiation site, e.g., between −10 and +10 regions of a gene encoding a polypeptide according to the invention, are preferred. Ribozymes are enzymatic RNA molecules capable of catalysing the specific cleavage of RNA. The mechanism of ribozyme action involves sequence specific hybridization of the ribozyme molecule to complementary target RNA, followed by a endonucleolytic cleavage. Within the scope of the invention are engineered hammerhead motif ribozyme molecules that specifically and efficiently catalyse endonucleolytic cleavage of adhesin-encoding RNA sequences.

Specific ribozyme cleavage sites within any potential RNA target are initially identified by scanning the target molecule for ribozyme cleavage sites, which include the following sequences, GUA, GUU and GUC. Once identified, short RNA sequences of between 15 and 20 ribonucleotides corresponding to the region of the target gene containing the cleavage site may be evaluated for predicted structural features such as secondary structure that may render the oligonucleotide sequence unsuitable. The suitability of candidate targets may also be evaluated by testing their accessibility to hybridization with complementary oligonucleotides, using ribonuclease protection assays.

Both anti-sense RNA and DNA molecules and ribozymes may be prepared by any method known in the art for the synthesis of RNA molecules. These include techniques for chemically synthesizing oligodeoxyribonucleotides well known in the art such as for example solid phase phosphoramidite chemical synthesis. Alternatively, RNA molecules may be generated by in vitro and in vivo transcription of DNA sequences encoding the antisense RNA molecule. Such DNA sequences may be incorporated into a wide variety of vectors which incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines.

Various modifications to the DNA molecules may be introduced as a means of increasing intracellular stability and half-life. Possible modifications include but are not limited to the addition of flanking sequences of ribo- or deoxy-nucleotides to the 5′ and/or 3′ ends of the molecule or the use of phosphorothioate or 2′ O-methyl rather than phosphodiesterase linkages within the oligodeoxyribonucleotide backbone.

Alternatively, an adhesin inhibitor may comprise an antigen-binding molecule that is immuno-interactive with an extracellular portion of the adhesin of the invention. Such antigen-binding molecules can be produced by any suitable method known to persons of skill in the art. Examples of antigen-binding molecules, which are contemplated by the present invention, include those described in Section 5.2.1.

8. Compositions and Therapeutic/Prophylactic Interventions

The adhesin polypeptides, fragments, variants and derivatives, and the polynucleotides and polynucleotide variants described in Section 3, and the modulatory agents described in Section 6 and 7 (therapeutic agents) can be used as actives for the treatment or prophylaxis of male fertility or for enhancing the propensity of a favorable pregnancy outcome, or for enhancing the propensity of a favorable ART outcome as, for example, described infra. These therapeutic agents can be administered to a patient either by themselves, or in pharmaceutical compositions where they are mixed with a suitable pharmaceutically acceptable carrier. Alternatively, the therapeutic agents may be used to treat a sperm sample of a patient. The patient includes, but is not restricted to, a sperm donor, an oocyte donor, a sperm recipient, an oocyte recipient and an embryo recipient.

Accordingly, the invention also provides a composition for enhancing or otherwise improving male fertility, or for enhancing the propensity for a favorable pregnancy outcome, or for enhancing the propensity for a favorable assisted reproductive technology (ART) outcome, wherein said composition comprises an agent that reduces the level and/or functional activity of an adherovar adhesin, and optionally a pharmaceutically acceptable carrier.

The invention further provides a method for improving male fertility, enhancing the propensity for a favorable pregnancy outcome, or for enhancing the propensity for a favorable assisted reproductive technology (ART) outcome in a patient. The method comprises administering to a patient, or to the patient's sperm donor, as the case may be, or to the sperm of a patient or to the sperm of the patient's sperm donor, as the case may be, an effective amount of an agent that reduces the level and/or functional activity of an adherovar adhesin of the invention, and optionally a pharmaceutically acceptable carrier.

Therapeutic agents may be formulated and administered systemically or locally. Techniques for formulation and administration may be found in “Remington's Pharmaceutical Sciences,” Mack Publishing Co., Easton, Pa., latest edition. Suitable routes may, for example, include oral, rectal, transmucosal, or intestinal administration; parenteral delivery, including intramuscular, subcutaneous, intramedullary injections, as well as intrathecal, direct intraventricular, intravenous, intraperitoneal, intranasal, or intraocular injections. For injection, the therapeutic agents of the invention may be formulated in aqueous solutions, preferably in physiologically compatible buffers such as Hanks' solution, Ringer's solution, or physiological saline buffer. For transmucosal administration, penetrants appropriate to the barrier to be permeated are used in the formulation. Such penetrants are generally known in the art. Intra-muscular and subcutaneous injection is appropriate, for example, for administration of immunogenic compositions, vaccines and DNA vaccines.

The agents can be formulated readily using pharmaceutically acceptable carriers well known in the art into dosages suitable for oral administration. Such carriers enable the compounds of the invention to be formulated in dosage forms such as tablets, pills, capsules, liquids, gels, syrups, slurries, suspensions and the like, for oral ingestion by a patient to be treated. These carriers may be selected from sugars, starches, cellulose and its derivatives, malt, gelatine, talc, calcium sulphate, vegetable oils, synthetic oils, polyols, alginic acid, phosphate buffered solutions, emulsifiers, isotonic saline, and pyrogen-free water.

Pharmaceutical compositions suitable for use in the present invention include compositions wherein the active ingredients are contained in an effective amount to achieve its intended purpose. The dose of agent administered to a patient should be sufficient to effect a beneficial response in the patient over time such as improving male fertility or enhancing the propensity of a favorable pregnancy outcome, or for enhancing the propensity of a favorable ART outcome. The quantity of the agent(s) to be administered may depend on the subject to be treated inclusive of the age, sex, weight and general health condition thereof. In this regard, precise amounts of the agent(s) for administration will depend on the judgement of the practitioner. In determining the effective amount of the agent to be administered in the treatment or prophylaxis of the condition, the physician may evaluate tissue levels of a polypeptide, fragment, variant or derivative of the invention, and progression or amelioration of the condition or disorder. In any event, those of skill in the art may readily determine suitable dosages of the therapeutic agents of the invention.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Pharmaceutical preparations for oral use can be obtained by combining the active compounds with solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Such compositions may be prepared by any of the methods of pharmacy but all methods include the step of bringing into association one or more therapeutic agents as described above with the carrier which constitutes one or more necessary ingredients. In general, the pharmaceutical compositions of the present invention may be manufactured in a manner that is itself known, e.g., by means of conventional mixing, dissolving, granulating, dragee-making, levigating, emulsifying, encapsulating, entrapping or lyophilizing processes.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterise different combinations of active compound doses.

Pharmaceutical which can be used orally include push-fit capsules made of gelatin, as well as soft, sealed capsules made of gelatin and a plasticiser, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added.

Dosage forms of the therapeutic agents of the invention may also include injecting or implanting controlled releasing devices designed specifically for this purpose or other forms of implants modified to act additionally in this fashion. Controlled release of an agent of the invention may be effected by coating the same, for example, with hydrophobic polymers including acrylic resins, waxes, higher aliphatic alcohols, polylactic and polyglycolic acids and certain cellulose derivatives such as hydroxypropylmethyl cellulose. In addition, controlled release may be effected by using other polymer matrices, liposomes and/or microspheres.

Therapeutic agents of the invention may be provided as salts with pharmaceutically compatible counterions. Pharmaceutically compatible salts may be formed with many acids, including but not limited to hydrochloric, sulphuric, acetic, lactic, tartaric, malic, succinic, etc. Salts tend to be more soluble in aqueous or other protonic solvents that are the corresponding free base forms.

For any compound used in the method of the invention, the therapeutically effective dose can be estimated initially from cell culture assays. For example, a dose can be formulated in animal models to achieve a circulating concentration range that includes the IC50 as determined in cell culture (e.g., the concentration of a test agent, which achieves a half-maximal inhibition or enhancement of ureaplasma adherence to sperm). Such information can be used to more accurately determine useful doses in humans.

Toxicity and therapeutic efficacy of such therapeutic agents can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit large therapeutic indices are preferred. The data obtained from these cell culture assays and animal studies can be used in formulating a range of dosage for use in human. The dosage of such compounds lies preferably within a range of circulating concentrations that include the ED50 with little or no toxicity. The dosage may vary within this range depending upon the dosage form employed and the route of administration utilised. The exact formulation, route of administration and dosage can be chosen by the individual physician in view of the patient's condition. (See for example Fingl et al., 1975, in “The Pharmacological Basis of Therapeutics”, Ch. 1, p. 1).

Dosage amount and interval may be adjusted individually to provide plasma levels of the active agent which are sufficient to maintain ureaplasma adhesin-inhibitory or enhancement effects. Usual patient dosages for systemic administration range from 1-2000 mg/day, commonly from 1-250 mg/day, and typically from 10-150 mg/day. Stated in terms of patient body weight, usual dosages range from 0.02-25 mg/kg/day, commonly from 0.02-3 mg/kg/day, typically from 0.2-1.5 mg/kg/day.

Alternately, one may administer the agent in a local rather than systemic manner, for example, via injection of the compound directly into a tissue, which is preferably a testicular tissue, often in a depot or sustained release formulation. Furthermore, one may administer the agent in a targeted drug delivery system, for example, in a liposome coated with tissue-specific antibody. The liposomes will be targeted to and taken up selectively by the tissue.

In another embodiment, the therapeutic agents of the invention can be used for treatment in vitro of spermatozoa or oocytes that are infected with ureaplasma. For example, during their preparation for an artificial reproductive technique, the spermatozoa and/or oocytes can be treated in vitro with a therapeutic agent of the invention. If desired, the spermatozoa and/or oocytes may also be treated with other agents including antibiotics, which are effective against adherovars.

From the foregoing, it will be appreciated that the agents of the invention may be used as therapeutic or prophylactic immunomodulating compositions or vaccines. Accordingly, the invention extends to the production of immunomodulating compositions for eliciting an immunogenic response, and particularly the production of elements that specifically bind to the adherovars of the invention. The composition comprises a proteinaceous molecule selected from the group consisting of an isolated adhesin of said adherovar, a biologically active fragment of said adhesin, a variant of said adhesin, a variant of said biologically active fragment, a derivative of said adhesin, a derivative of said biologically active fragment, and a derivative of said variant, and/or or a vector comprising a polynucleotide encoding said proteinaceous molecule, wherein said polynucleotide is operably linked to a regulatory polynucleotide, and wherein said composition optionally comprises one or both of a pharmaceutically acceptable carrier or adjuvants.

The invention also extends to a method for treatment and/or prophylaxis of male infertility, an adverse pregnancy outcome or an adverse assisted reproductive technology (ART) outcome in a patient, comprising administering to the sperm donor or to the patient's sperm donor, as the case may be, an immunogenically effective amount of the immunopotentiating composition as broadly described above.

Any suitable procedure is contemplated for producing the immunomodulating compositions of the invention. Exemplary procedures include, for example, those described in NEW GENERATION VACClNES (1997, Levine et al., Marcel Dekker, Inc. New York, Basel, Hong Kong).

It will be appreciated from the foregoing that the present invention contemplates the use of nucleic acid compositions for the purpose of vaccination or immunomodulation. In this regard, a synthetic construct can be used to immunise a patient, which construct includes a polynucleotide encoding an adhesin according to the invention, wherein said polynucleotide is operably connected to one or more regulatory sequences that direct expression of said polynucleotide in said patient.

Typically, such constructs or vectors are derived from viral DNA sequences such as adenovirus, adeno-associated viruses, herpes-simplex viruses and retroviruses. Suitable immunomodulating vectors currently available to the skilled person may be found, for example, in Wu and Ataai (2000, Curr. Opin. Biotechnol. 11 (2): 205-208), Vigna and Naldini (2000, J. Gene Med. 2 (5): 308-316), Kay, et al. (2001, Nat. Med. 7 (1): 33-40), Athanasopoulos, et al. (2000, Int. J. Mol. Med. 6 (4): 363-375) and Walther and Stein (2000, Drugs 60 (2): 249-271).

Administration of the immunomodulating construct to a patient, preferably a human patient, may include delivery via direct oral intake, systemic injection, or delivery to selected tissue(s) or cells, or indirectly via delivery to cells isolated from the patient or a compatible donor. In a preferred embodiment, the immunomodulating construct is delivered intradermally. Delivery of said immunomodulating construct to cells or tissues of the patient or said compatible donor may be facilitated by microprojectile bombardment, liposome mediated transfection (e.g., lipofectin or lipofectamine), electroporation, calcium phosphate or DEAE-dextran-mediated transfection, for example. A discussion of suitable delivery methods may be found in Chapter 9 of CURRENT PROTOCOLS IN MOLECULAR BIOLOGY (Eds. Ausubel et al.; John Wiley & Sons Inc., 1997 Edition), for example, which is herein incorporated by reference.

The step of introducing the immunomodulating construct into a target cell or tissue will differ depending on the intended use and species, and can involve one or more of non-viral and viral vectors, cationic liposomes, retroviruses, and adenoviruses such as, for example, described in Mulligan, R. C., (1993). Such methods can include, for example:
A. Local application of the expression vector by injection (Wolff et al., 1990), surgical implantation, instillation or any other means. This method can also be used in combination with local application by injection, surgical implantation, instillation or any other means, of cells responsive to the protein encoded by the expression vector so as to increase the effectiveness of that treatment. This method can also be used in combination with local application by injection, surgical implantation, instillation or any other means, of another factor or factors required for the activity of said protein.
B. General systemic delivery by injection of DNA, (Calabretta et al., 1993), or RNA, alone or in combination with liposomes (Zhu et al., 1993), viral capsids or nanoparticles (Bertling et al., 1991) or any other mediator of delivery. Improved targeting might be achieved by linking the polynucleotide/expression vector to a targeting molecule (the so-called “magic bullet” approach employing, for example, an antigen-binding molecule), or by local application by injection, surgical implantation or any other means, of another factor or factors required for the activity of the protein encoded by said expression vector, or of cells responsive to said protein.
C. Injection or implantation or delivery by any means, of cells that have been modified ex vivo by transfection (for example, in the presence of calcium phosphate: Chen et al., 1987, or of cationic lipids and polyamines: Rose et al., 1991), infection, injection, electroporation (Shigekawa et al., 1988) or any other way so as to increase the expression of said polynucleotide in those cells. The modification can be mediated by plasmid, bacteriophage, cosmid, viral (such as adenoviral or retroviral; Mulligan, 1993; Miller, 1992; Salmons et al., 1993) or other vectors, or other agents of modification such as liposomes (Zhu et al., 1993), viral capsids or nanoparticles (Bertling et al., 1991), or any other mediator of modification. The use of cells as a delivery vehicle for genes or gene products has been described by Barr et al., 1991 and by Dhawan et al., 1991. Treated cells can be delivered in combination with any nutrient, growth factor, matrix or other agent that will promote their survival in the treated subject.

Immunomodulating compositions according to the present invention can also contain a physiologically acceptable diluent or excipient such as water, phosphate buffered saline and saline. They may also include an adjuvant as is well known in the art. Suitable adjuvants include, but are not limited to: surface active substances such as hexadecylamine, octadecylamine, octadecyl amino acid esters, lysolecithin, dimethyldioctadecylammonium bromide, N,N-dicoctadecyl-N′,N′bis(2-hydroxyethyl-propanediamine), methoxyhexadecylglycerol, and pluronic polyols; polyamines such as pyran, dextransulfate, poly IC carbopol; peptides such as muramyl dipeptide and derivatives, dimethylglycine, tuftsin; oil emulsions; and mineral gels such as aluminum phosphate, aluminum hydroxide or alum; lymphokines, QuilA and immune stimulating complexes (ISCOMS).

In order that the invention may be readily understood and put into practical effect, particular preferred embodiments will now be described by way of the following non-limiting examples.

### EXAMPLES

### Example 1

Patients

This prospective study investigated couples participating in ART procedures at The Wesley IVF Service, The Wesley Hospital, Queensland; Australia between September 1999 and May 2002. All consenting couples presenting for a fully stimulated ovarian treatment cycle or a minimal stimulated cycle were enrolled in the project. Stimulation and growth of ovarian follicles in a fully stimulated cycle was achieved with subcutaneous injections of FSH (follicle stimulating hormone) (Puregeon, Organon, Sydney, Australia or Gonalf, Serono Laboratories, Sydney Australia) and nasal application of GnRH analogues to suppress ovulation (Leucrin, Abbotts, Sydney, Australia or Synarel, Monsarto, Sydney, Australia). The growth of the ovarian follicles was monitored by vaginal ultrasound scans every second or third day. Finally the administration of 10,000 U hCG (Profasi, Serono Laboratories, Sydney, Australia) stimulated the maturation and release of the ovarian follicles. Oocytes were retrieved transvaginally 36 hours after the HCG injection under ultrasound guidance

### Example 2

Endocervical Swabs

Female partner undergoes a stimulated cycle to trigger ovulation of increased numbers of oocytes. Prior to oocyte collection two endocervical swabs were collected during a speculum examination for subsequent ureaplasma culture. One swab was inoculated directly into 10B Broth (Shepard, 1978) and the second was placed in Stuart's transport media. All clinical samples were frozen at −80° C. until processing for culture and PCR.

### Example 3

Semen Sample Preparation

Semen samples were collected on the day of oocyte retrieval. The semen was collected by masturbation into a sterile container after three days of abstinence. Samples were collected at the IVF laboratory or delivered to the laboratory within 1 hour (preferably within 20 minutes) of collection. Prior to washing 100 μL of semen was inoculated into 1.8 mL of 10B broth for ureaplasma culture. The semen was then washed in a PureSperm™ (Genartech, Thornleigh, NSW, Australia) gradient (Mortimor, 1994) consisting of 1 mL 95% PureSperm solution [prepared using culture medium consisting of modified human tubal fluid (HTF) (Irvine Scientific, Santa Ana, Calif.) supplemented with either 4 mg/mL Albumex or 10% prepared autologous patients' serum]; a 1 mL overlay of 47.5% PureSperm; and an overlay of 1 mL of raw semen. The gradient was centrifuged (Beckman, Allegra-6) for 15 min at 1000 rpm (300×g). The pellet was subsequently washed twice in 2-3 mL of culture medium and centrifuged for 10 minutes at 1000 rpm (300×g). The washed spermatozoa was resuspended in culture medium to a final concentration of <25 million/mL for in vitro fertilization (IVF) insemination or fertilization by ICSI. One hundred μL of the washed semen was also inoculated into 1.8 mL of 10B broth for ureaplasma culture. Additionally 20 μL of semen and washed semen were placed on separate wells on a Multitest Slide (ICN, Aurora, Ohio, USA), allowed to air dry and then fixed in cold acetone for 1 minute. These slides were tested immediately or stored at −20° C. until testing.

For some patients the spermatozoa were collected by testicular aspiration or alternatively sections of the seminiferous tubules were removed and the sperm was extracted from this tissue (Allan, 1997). This spermatozoa was washed in 80% PureSperm™ prior to ICSI fertilization (Allan, 1997). Testicular sperm remaining after the completion of the ART procedure was inoculated into 10B broth. All 10B broth specimens were frozen at −80° C. until processing.

### Example 4

Testicular Spermatozoa

If desired, the adherovars of the invention, especially ureaplasma, can be detected in testicular spermatozoa, which can be collected by testicular aspiration or alternatively sections of the seminiferous tubules can be removed and the sperm was extracted from this tissue (Allan, 1997). These spermatozoa are then rinsed in modified HTF and centrifuged for 15 min at 1500 rpm (400×g). The pellet is resuspended in 1 mL of patient Flush medium, gently pipetted to disrupt the tissue, layered over 0.5 mL of 80% PureSperm™ then centrifuged for 10 min at 1500 rpm. The pellet is resuspended in flush fluid and motile sperm (twitching) were then selected for ICSI fertilization (Allan, 1997). Testicular sperm remaining after the completion of the ART procedure is then inoculated into 10B broth. All 10B broth specimens were frozen at −80° C. until processing.

### Example 5

Swim-Up Method of Sperm Preparation.

This preparation protocol is used for semen samples respond poorly to PureSperm™ preparation, or have severe clumping that is exacerbated by PureSperm™ preparation (Osborn)

Sterile 5 mL test tubes are wetted with HTF/4 mg/mL albumex™ and then 0.5 mL of semen is placed in each tube. Then 0.5 mL of HTF/albumex™ solution is carefully layered over the top of the semen (a clean interface should be created). The tubes are capped loosely and placed in the incubator for 40 min and during this time motile sperm should swim up into the HTF/albumex™ solution. If adequate numbers of sperm are present in this layer then this is the final preparation for IVF or ICSI fertilization. However if the sperm count is low then the HTF/albumex™ layer from all tubes should be combined and centrifuged for 10 min at 1000 rpm (300×g). The supernatant is then removed and the pellet resuspended in 1-2 mL of culture medium.

### Example 6

Urea Plasma Culture

Prior to culture, two aliquots (0.5 mL) of each original specimen were stored at −80° C. All clinical samples (in 10B broth) then were serially diluted (three serial 10-fold dilutions) in 10B broth. Broths were cultured aerobically, at 37° C. until ureaplasma growth was detected by an alkaline shift and subsequent colour change in the media for 1 week. All positive semen samples were subsequently quantitated by re-culturing in eight serial 10-fold dilutions of 10B broth. Aliquots of ureaplasma positive broth cultures were also stored at −80° C.

### Example 7

DNA Extraction

Ureaplasma DNA was prepared by the method of Blanchard et al. (1993). Stored clinical samples (10B broth) of ureaplasmas were thawed and 500 μL of the sample was centrifuged at 15,900×g (Beckman Microfuge E) for 20 min at 4° C. The pellet was resuspended in 60 μL of Solution A (10 mM Tris HCl pH 8.3, 100 mM KCl, 2.5 mM MgCl₂) and 60 μL Solution B (10 mM Tris HCl pH 8.3, 2.5 mM MgCl₂, 1% Tween 20, 1% Triton X-100) with 120 μg/mL of proteinase K. The suspension was incubated for 1 hour at 60° C., then 10 min at 94° C., then cooled and 5-20 μL of each sample was used for mba gene PCR or stored at −20° C. until PCR analysis (Blanchard, A., Gautier, M. and Mayau, V., 1991).

### Example 8

PCR Detection

All cultured samples were tested by PCR assays for ureaplasmas and M. hominis. The DNA was extracted using previously described methods (Knox and Timms, 1998). Ureaplasmas were detected and speciated using the mba primer pair UM 1 (UMS 125 and UMA 226) (Teng et al. 1993) and previously described PCR conditions (Knox, et al., 1998). All washed semen samples were further tested using a nested PCR reaction: outer primers UMS-125 and UMA 226; and inner primers UMS −9 (5′ ATTTTTTATATTAGGAG 3′; [SEQ ID NO: 1]) and UMA 163 (5′ TTCAATGTCGTAAAC 3′; [SEQ ID NO: 2]). All clinical samples were also tested by PCR for M. hominis using a 16S rRNA assay (Blanchard, 1993).

All ureaplasma isolates were further subtyped using mba PCR assays. U. parvum isolates were typed by four separate reactions using the primer pairs for mba (serovar) 1 (UMS −83 and UMA −41), mba (serovar) 3 (serovar 3 UMS −81 and UMA 144), and mba (serovar) 6 (UMS −53 and UMA 122) (Knox and Timms, 1998). mba (serovar) 14 was typed using primers serovar 14 UMS −81 (5′ AGAAATTATGTAAGATTAAT 3′; [SEQ ID NO: 3]) and UMA 144.

All U. urealyticum isolates were subtyped into five mba types by primers designed from a sequence alignment of the GenBank mba sequences of the ATCC strains of U. urealyticum serovars 2, 4, 5, 7-13. These five separate reactions used the upstream primer UMS −7 (5′ ATTTCATATTTAGTTTATTAGGSGATCG 3′; [SEQ ID NO: 4]) in combination with UMA 426 (5′ TTCCTGGTTGTGTTTCAAAACCTATA 3′; [SEQ ID NO: 5]) for mba type 2, 5, 8, 10 (serovars 2, 5, 8 and 10); UMA 429 (5′ TGCCTGGTTGTGTTTCGAAACTCC 3′; [SEQ ID NO: 6]) for mba type 4, 2, 13 (serovars 4, 12 and 13); UMA 440 (5′ CGTTGGTTCTGGTGTATGAGTTGC 3′; [SEQ ID NO: 7]) for mba 7,11 (serovars 7 and 11); UMA 442 (5′ GTTCTGGAGTTGGTGTAGGCGC 3′; [SEQ ID NO: 8]) for mba 9 (serovar 9); or UMA 461 (5′ TTGAACCACTTCCTGGTTGTGTAG 3′; [SEQ ID NO: 9]) for mba 10 (serovar 10).

Separate PCRs were performed for each primer pair. The reaction mixture (50 μL) contained final concentrations of 2.5 U of HotStar™ Taq (Qiagen, Clifton Hill, Australia), 1×PCR buffer (Tris-Cl, KCl, (NH₄)₂SO₄, 15 mM MgCl₂, pH 8.7 (20° C.) (Qiagen, Clifton Hill, Australia), 1×Q solution (Qiagen, Clifton Hill, Australia), 200 μM each deoxynucleotide triphosphate (Roche, Castle Hill, Australia), distilled water, 0.5 μM of each primer and DNA template (5-15 μL of prepared sample). The PCRs were individually optimised and the reaction parameters for each primer pair were critical. The DNA thermal cycler 480 (Perkin-Elmer Cetus) was programmed for: 1 cycle of denaturation, 94° C. for 15 min followed by 35 cycles consisting of denaturation at 94° C. for 1 min, annealing for 1 min (UM1 primer pair −55° C., nested inner primers −36° C., M. hominis primers −55° C., serovar 1-57° C.; serovar 3 and 14-52° C.; serovar 6-56° C.; serovars 2, 4, 5 7-13-65° C.). This was followed by a final cycle of 72° C. for 10 min. PCR products were separated by electrophoresis in a 2% agarose gel and visualised by ethidium bromide staining and digitised using Grab-IT (Ultraviolet Products LTD, Cambridge, England, United Kingdom). Reference serovars 1, 3, 6, 8 and 14 and ATCC strains for serovars 2, 4, 5, 7, 9-13 were used as controls.

### Example 9

Indrect IFA Testing of Semen and Washed Semen Samples

The fixed slides of undiluted semen and washed semen initially were blocked for 1 hour with a mixture of 10% skim milk, 70% horse serum (CSL, Brisbane, Australia), 10% BSA and 10% gelatin. The slides then were incubated with the primary antibody (serovar specific for U. parvum and pooled serovars for U. urealyticum), rabbit anti-ureaplasma antiserum (1/100 dilution in PBS) which was kindly provided by Dr Patricia Quinn (Department of Bacteriology, The Hospital for Sick Children, Toronto General Hospital, Canada). The slides were then washed in PBS (3×5 mins), incubated with the secondary goat anti-rabbit Alex Fluor 568 (Molecular Probes, Eugene, Oreg., USA), washed again in PBS, mounted and then viewed using confocal microscopy (Leica TCS 4D Confocal laser scanning microscope).

### Example 10

Prevalence of ureaplasma Subtypes in Clinical Samples

Clinical samples for ureaplasma culture and PCR detection were collected from 319 couples participating in ART treatment cycles. Ureaplasmas were detected in: 22% (70/319) of semen samples; 9.1% (29/319) of washed semen samples; and 37% (118/319) of endocervical samples. All clinical ureaplasma isolates were subtyped using PCR assays (TABLE I). U. parvum isolates were detected more frequently in both men (70% of isolates) and women (91.5% of isolates). However there was a higher incidence of U. urealyticum in men (18% of isolates) than in women (8.5% of isolates). Mixed serovars were detected in 8/319 semen samples (2.5%) and 20/319 endocervical samples (6.3%). In 9/319 (2.8%) couples the male and female partners were found to be infected/colonized with different ureaplasma subtypes. U. parvum serovar 3 was isolated most frequently from the endocervix of female partners (36%), and from male semen (36%). However, U. parvum serovar 6 is the most prevalent subtype in male washed semen (37.9%). Serovar 6, has a stronger in vivo binding affinity for spermatozoa than serovars 1 and 3, and was not removed by washing in a pure sperm 95/47.5 gradient procedure prior to an ART treatment in 50% of men colonized/infected with serovar 6. By contrast 83% of serovar 1 and 78.6% of serovar 3 clinical isolates were removed from the spermatozoa by this standard washing procedure. Of the 14 U. urealyticum (10 different serovars) isolates detected in semen, 9 (64%) were not removed by this ART washing procedure.

In this study M. hominis has been detected in 3/105 (2.8%) of semen samples tested. Washing of semen failed to remove this mycoplasma from 2/3 (66%) infected semen samples. M. hominis has also been detected in 2/101 (2%) of endocervical samples tested to date.

### Example 11

Indirect IFA Testing of Semen and Washed Semen Samples

Confocal microscopic examination of spermatozoa and washed spermatozoa, culture and PCR positive for ureaplasmas detected indirect IFA staining of ureaplasmas on the surface of the acrosome. Pleomorphic immunofluorescent forms consistent in size and morphology with ureaplasmas (as observed by TEM) were also observed free within the seminal plasma of some patients

### Example 12

Comparison of Fertilization Rates for 259 Couples with ureaplasma Positive Semen/Washed; ureaplasma Positive Semen/Washed Semen Negative and ureaplasma Semen Negative Couples.

The fertilization rates for ureaplasma washed semen positive couples and ureaplasma washed semen negative couples (two separate ureaplasma classifications: sperm positive; sperm negative) were compared (Table II). Overall a higher rate of oocyte fertilization was achieved using the ICSI procedure (average 69.2%) than by conventional IVF (average 65.8%). However, these data show a reduced IVF fertilization rate for ureaplasma washed sperm positive couples (60.1%) compared to semen positive washed sperm negative couples (71.69%) and ureaplasmas semen/washed semen negative couples (66%).

### Example 13

Comparison of the Total Number of Pregnancies Achieved by Fresh and Frozen Embryo Transfer after Fertilization by Either IVF or ICSI Techniques

Pregnancy outcome data (Table III) for all patients after IVF and CISI insemination procedures and fresh and frozen embryo transfer shows a reduced viable pregnancy rate in couples with ureaplasma positive washed semen (9%) compared to those couples with ureaplasma positive sperm/negative washed sperm (23.5%) and couples with ureaplasma negative sperm/washed sperm (14%). However if we consider the pregnancy outcomes for IVF and ICSI pregnancies separately (Table IV and Table V) a greatly reduced viable pregnancy rate is observed for ureaplasma washed sperm positive ICSI couples (5%) but not for ureaplasma positive washed sperm IVF couples (13%)

There was an increased miscarriage rate in couples with ureaplasma positive washed sperm (56%) compared to couples with ureaplasma negative washed sperm (20% and 27% respectively). An increased miscarriage rate is demonstrated in both IVF (50%) and ICSI (66%) ureaplasma washed sperm positive couples when compared to: ureaplasma positive sperm/negative washed sperm couples (20%, 20% respectively); and ureaplasma sperm/washed sperm negative couples (35.5%, 26% respectively) (Table IV and Table V). By contrast there were no real differences in the viable pregnancy rate (15%) and miscarriage rate (28%) of ureaplasma endocervical positive couples compared to ureaplasma endocervical negative couples (13% and 37.5% respectively) (Table VI).

Conclusions

Not wishing to be bound by any one particular theory or mode of operation, the inventors believe that the mechanism by which infection of the embryo and the placenta are initiated is central to elucidating the pathogenesis of adherovars, and especially of the ureaplasmas, in adverse pregnancy outcome. In this light, it is proposed that adherovar infection of the embryo and the placenta occurs as a result of fertilization of an oocyte by spermatozoa with adherent adherovars and the results from this current study add further support to this hypothesis. To summarise:

1. Ureaplasmas attached to the surface of the acrosome of spermatozoa have been observed by confocal microscopy after indirect WFA staining.

2. Furthermore, the inventors have also demonstrated (by culture and PCR assays) that ureaplasmas were not removed from 41.5% of infected/colonized semen samples by washing in a Pure sperm 95/47.5 gradient and this confirms there is a risk that the embryo may become infected during ART fertilization procedures.

3. Significantly the present inventors have also shown that U. parvum serovar 6 has a stronger in vivo binding affinity for spermatozoa and were not removed from semen in 50% of those infected/colonized with this serovar. By contrast U. parvum serovar 1 was the least adherent to spermatozoa and was removed from 83% of ureaplasma positive semen samples by the washing procedure.

4. This current study has also confirmed that ureaplasma positive semen does not affect the fertilization rate using either ICSI of IVF techniques. However the inventors have shown a slightly reduced IVF fertilization rate (but not the ICSI rate) in couples with ureaplasma positive washed semen (60.1%) compared to couples with ureaplasma negative washed semen (average 67.6%) (Table II). Previously Busolo and Zanchetta (1984) (Busolo and Zanchetta, 1984) demonstrated reduced oocyte penetration rates by spermatozoa pre-incubated with ureaplasmas (serovars 1-8). The greatest reductions in oocyte penetration were observed for spermatozoa pre-incubated with serovar 6. The least reduction in penetration was observed for serovar 1—the serovar, which was less adherent to spermatozoa and removed more frequently by washing in this current study. These results suggest that the binding affinity of the different ureaplasma serovars to spermatozoa could be an important determinant affecting oocyte penetration and fertilization.

5. The present investigation has also demonstrated a reduced viable pregnancy rate in ureaplasma positive washed semen ICSI couples and an increased miscarriage rate in all couples with ureaplasma positive washed semen, but not in couples with ureaplasma positive semen/negative washed semen or in couples with ureaplasma positive endocervical samples (Table III, IV and V). During ICSI fertilization techniques, ureaplasmas adherent to spermatozoa in washed semen samples are injected into and infect the oocyte. In ICSI patients there is only one continuing viable pregnancies (1/21 (5%) embryo transfers) and the results herein reveal a 66% (2/3 pregnancies) miscarriage rate in ureaplasma washed sperm positive couples. There is evidence that the oocyte also becomes infected during IVF procedures. In ureaplasma washed semen positive IVF patients there is a 13% viable pregnancy rate and a 50% miscarriage rate. Whilst these rates are still different to ureaplasma negative washed semen couples, it appears that natural selective processes may in some instances facilitate fertilization by spermatozoa without adherent ureaplasmas and thus prevent infection at conception. Whilst U. parvum serovar 6 was isolated from the washed semen of all ICSI couples (ureaplasma washed semen positive couples) who miscarried, adherent U. parvum 6 (1 couple) and adherent U. urealyticum (2 couples) were isolated from the washed semen of IVF couples who miscarried. Furthermore no pregnancies were achieved in couples with M. hominis positive washed semen.

The above results strongly support the hypothesis that adherovar, especially ureaplasma, adherence to spermatozoa is a mechanism of pathogenicity facilitating infection of the embryo at conception. The teachings described herein have implications for the future treatment of not only ART patients but also couples with a recurrent history of preterm delivery due to adherovar chorioamnionitis. Screening of couples prior to an ART treatment cycles will identify couples with adherovars in washed semen samples. Antibiotic treatment of both partners just prior to oocyte retrieval could eradicate adherovars and improve ART pregnancy outcomes.

### Example 14

Identification of Adhesin—U. parvum Serovar 6

Interactions of whole cell ureaplasmas (previously characterised as adherent and non-adherent, in vivo and in vitro) with SGG (purified from bull testes, Lingwood et al. 1980) and SCG (Sigma) are compared using real-time biomolecular interaction analysis (BIA) in a BIAcore™ 2000, a system that uses surface plasmon resonance (SPR). The SGG (or SCG) is bound to the surface of a sensor chip. Briefly, liposomes of dimyristoylphosphatidylcholine in buffer are seeded (1-5%) with SGG, and are prepared by extrusion through 50 nm pore size filters. The liposomal mixture (the ligand) is allowed to flow through the BIAcore™ 2000 and is bound to LI sensor chips (BIAcore™ AB, Australia). Whole cell ureaplasmas (analytes) in solution are run over this surface and the interaction is followed in real time by SPR analysis. Low flow rates and sample recovery are used to ensure whole cells are not lysed by these procedures. The advantages of using this system are that: whole cell ureaplasmas can be used; interactions are followed in real time without labelling of either the cells or the immobilized ligand; the relative affinities of different ureaplasma serovars for either ligand can be determined; and the kinetics of the different interactions of different ureaplasma serovars directly compared. Mixtures of clinical ureaplasma serovars can also be analyzed to determine if there is preferential binding to the ligands of particular serovars from a mixed population. The preferentially bound serovars will be identified after sample recovery.

Cell lysates and membrane preparations from (clinically defined) strongly-adherent and non-adherent serovars are compared for their ability to interact with SCG in the BIAcore™ 2000. Whole cell ureaplasmas are pre-treated with proteases (papain, trypsin, chymotrypsin) or heat in the presence of reductants (β-mercaptoethanol) to determine if the SGG receptor is proteinaceous. Preparations of the proteinaceous receptor are suitably examined by SDS-PAGE for any obvious differences in protein composition, but, importantly, the recovery option of the BIAcore™ 2000 is used to capture any specifically bound material for similar examination. Recovered material, either from BIAcore™ or excised from PVDF membrane following Western blot is N-terminally sequenced by Edman degradation, mass spectrometry or both.

In another example, trypsin or chymotrypsin pre-treatment of adherent ureaplasma cells is used to prevent adherence, and subsequently the proteolytic fragments produced by this treatment are examined for their ability to bind to SGG. If necessary, fractionation of these fragments by reverse-phase HPLC over C4 or C8 matrices is used to determine the identity of the fragment(s) specifically involved in such interactions. The fragments with a demonstrated binding ability to bind SGG are be characterised by N-terminal sequencing or mass spectrometry, or both, and compared to commercially available protein databases and the complete sequence of U. parvum serovar 3 (Glass et al. 2000) for identification.

In yet another example, an adhesin may be identified by comparing surface accessible membrane proteins of adherent (serovar 6) and non-adherent (serovar 1) ureaplasma isolates (first passage clinical isolates). A number of strategies known to persons of skill in the art can be employed in this regard.

The disclosure of every patent, patent application, and publication cited herein is hereby incorporated herein by reference in its entirety.

The citation of any reference herein should not be construed as an admission that such reference is available as “Prior Art” to the instant application.

Throughout the specification the aim has been to describe the preferred embodiments of the invention without limiting the invention to any one embodiment or specific collection of features. Those of skill in the art will therefore appreciate that, in light of the instant disclosure, various modifications and changes can be made in the particular embodiments exemplified without departing from the scope of the present invention. All such modifications and changes are intended to be included within the scope of the appended claims.

Tables

TABLE 1
Prevalence of ureaplasma subtypes in clinical samples from 319 patients

| Ureaplasma subtypes | Semen | Washed Semen | Endocervical swabs |
|---|---|---|---|
| U. parvum serovar 1 | 12 | 2 | 18 |
| U. parvum serovar 3 | 28 | 6 | 51 |
| U. parvum serovar 6 | 22 | 11 | 38 |
| U. parvum serovar 14 | 0 | 0 | 0 |
| Ureaplasma urealyticum | 14 | 9 | 12 |
| Untyped, U. parvum | 1 | 1 | 20 |
| No. of isolates | 77/70 | 29/29 samples | 141/118 samples |
| | samples | | |

TABLE II
Comparison of fertilization rates for 259 couples with ureaplasma positive semen/washed;
ureaplasma positive semen/washed semen negative and ureaplasma semen negative couples.

| | Ureaplasma | | Ureaplasma | | Ureaplasma | | | |
|---|---|---|---|---|---|---|---|---|
| Method of | Washed Sperm +ve | | Washed sperm −ve | | Washed sperm −ve | | | |
| fertilization | Sperm +ve | | Sperm +ve | | Sperm −ve | | Total | |
| ICSI fertilization | 84/124 | 67.8% | 116/159 | 72.96% | 638/929 | 68.7% | 855/1235 | 69.2% |
| rate* | | | | | | | | |
| IVF fertilization | 95/158 | 60.1% | 81/113 | 71.69% | 433/654 | 66% | 609/925 | 65.8% |
| rate^{†} | | | | | | | | |
| Total rate of | 179/282 | 63.48% | 197/272 | 72.46% | 1071/1583 | 67.6% | 1464/2160 | 67.6% |
| fertilization | | | | | | | | |

*no. fertilized oocytes/no. injected with spermatozoa,
^{†}no. fertilized oocytes/total no. of oocytes retrieved

TABLE III
A comparison of the total number of pregnancies achieved by fresh
and frozen embryo transfer (ET) (412 ET) after fertilization by either
IVF or ICSI techniques for couples with ureaplasma positive
semen/washed; ureaplasma positive semen/washed semen negative
and ureaplasma semen negative couples.

| | Ureaplasma | | | | | |
|---|---|---|---|---|---|---|
| | Sperm +ve | | Ureaplasma | | Ureaplasma | |
| | Washed | | Sperm +ve | | Sperm −ve | |
| | sperm +ve | | Washed sperm −ve | | Washed sperm −ve | |
| TOTAL | ET = 44 | | ET = 51 | | ET = 317 | |
| Total preg/ET | 9 | 20.5% | 15 | 29% | 69 | 22% |
| Viable preg/ET | 4 | 9% | 12 | 23.5% | 44 | 14% |
| Miscarriages/ | 5/9 | 56% | 3/15 | 20% | 19/69 | 27% |
| Tot preg | | | | | | |
| Termination | | | | | 1 | |
| Ectopic | | | | | 3 | |

TABLE IV
A comparison of the total number of pregnancies achieved by fresh
and frozen embryo transfer (241 ET) after fertilization by ICSI techniques

| | Ureaplasma | | | | | |
|---|---|---|---|---|---|---|
| | Sperm +ve | | Ureaplasma | | Ureaplasma | |
| | Washed | | Sperm +ve | | Sperm −ve | |
| | sperm +ve | | Washed sperm −ve | | Washed sperm −ve | |
| ICSI TOTAL | ET = 21 | | ET = 35 | | ET = 185 | |
| Total preg/ET | 3 | 14% | 10 | 28.5% | 38 | 20.5% |
| Viable preg/ET | 1 | 5% | 8 | 23% | 26 | 14% |
| Miscarriages/ | 2/3 | 66% | 2/10 | 20% | 10/38 | 26% |
| Tot preg | | | | | | |
| Ectopic | | | | | 2 | |

TABLE V
A comparison of the total number of pregnancies achieved by fresh
and frozen embryo transfer (171 ET) after fertilization by IVF

| | Ureaplasma | | | | | |
|---|---|---|---|---|---|---|
| | Sperm +ve | | Ureaplasma | | Ureaplasma | |
| | Washed | | Sperm +ve | | Sperm −ve | |
| | sperm +ve | | Washed sperm −ve | | Washed sperm −ve | |
| IVF TOTAL | ET = 23 | | ET = 16 | | ET = 132 | |
| Total preg/ET | 6 | 26% | 5 | 31% | 31 | 23.5% |
| Viable preg/ET | 3 | 13% | 4 | 25% | 18 | 13.5% |
| Miscarriages/ | 3/6 | 50% | 1/5 | 20% | 11/31 | 35.5% |
| Tot preg | | | | | | |
| Termination | | | | | 1 | |
| Ectopic | | | | | 1 | |

TABLE VI
A comparison of the total number of pregnancies achieved by fresh and
frozen embryo transfer(412 ET) after fertilization by either IVF or ICSI
techniques for ureaplasma endocervical positive and negative couples

| | | |
|---|---|---|
| | Ureaplasma | Ureaplasma |
| | Endocervical +ve | Endocervical −ve |
| TOTAL | ET = 179 | ET = 233 |

| | | | | |
|---|---|---|---|---|
| Total preg/ET | 39 | 22% | 54 | 23% |
| Viable preg/ET | 27 | 15% | 33 | 14% |
| Miscarriages/Tot | 11/39 | 28% | 18/54 | 33% |
| preg | | | | |
| Termination | | | 1 | |
| Ectopic | 1 | | 2 | |

### BIBLIOGRAPHY

Allan, J. A., Cotman, A. S. (1997) A new method for freezing testicular biopsy sperm: three pregnancies with sperm extracted from cryopreserved sections of seminiferous tubule. Fertil Steril., 68, 741-744.
Blanchard, A., Gautier, M. and Mayau, V. (1991) Detection and identification of mycoplasmas by amplification of rDNA. FEMS Microbiol Letters., 81, 37-42.
Blanchard, A., Yanez, A., Dybvig, K., Watson, H. L., Griffiths, G., and Cassell, G. H. (1993) Evaluation of intraspecies genetic variation within the 16S rRNA gene of Mycoplasma hominis and detection by polymerase chain reaction. J Clin Microbiol., 31, 1358-1361.
Busolo, F. and Zanchetta, R. (1984) Do mycoplasmas inhibit the human sperm fertilizing ability in vitro? Isr J Med Sci., 20, 902-904.
Caspi, E., Herczeg, E., Solomon, F., et al. (1971) Amnionitis and T strain mycoplasmemia. Am J Obstet Gynecol., 111, 1102-1106.
Cassell, G. H., Davis, R. O., Waites, K. B., et al. (1983) Isolation of Mycoplasma hominis and Ureaplasma urealyticum from amniotic fluid at 16-20 weeks of gestation: potential effect on outcome of pregnancy. Sex Transm Dis., 10, 294-302.
Cassell, G. H., Waites, K. B., Gibbs, R. S., et al. (1986) Role of Ureaplasma urealyticum in amnionitis. Pediatr Infect Dis., 5, S247-252.
Cassell, G. H., Waites, K. B., Watson, H. L., et al. (1993) Ureaplasma urealyticum intrauterine infection: role in prematurity and disease in newborns. Clin Microbiol Rev., 6, 69-87.
Gray, D. J., Robinson, H. B., Malone, J., et al. (1992) Adverse outcome in pregnancy following amniotic fluid isolation of Ureaplasma urealyticum. Prenat Diagn., 12, 111-117.
Hill, A. C., Tucker, M. J., Whittingham, D. G., et al. (1987) Mycoplasmas and in vitro fertilization. Fertil Steril., 47, 652-655.
Kanakas, N., Mantzavinos, T., Boufidou, F., et al. (1999) Ureaplasma urealyticum in semen: is there any effect on in vitro fertilization outcome? Fertil Steril., 71, 523-527.
Keski-Nisula, L., Kirkinen, P., Katila, M. L., et al. (1997) Amniotic fluid U. urealyticum colonization: significance for maternal peripartal infections at term. Am J Perinatol., 14, 151-156.
Knox, C. L., Cave, D. G., Farrell, D. J., et al. (1997) The role of Ureaplasma urealyticum in adverse pregnancy outcome. Aust N Z J Obstet Gynaecol., 37, 45-51.
Knox, C. L., Giffard, P. and Timms, P. (1998) The phylogeny of Ureaplasma urealyticum based on the mba gene fragment. Int J Syst Bacteriol., 48, 1323-1331.
Knox, C. L. and Timms, P. (1998) Comparison of PCR, nested PCR, and random amplified polymorphic DNA PCR for detection and typing of Ureaplasma urealyticum in specimens from pregnant women. J Clin Microbiol., 36, 3032-3039.
Kong, F., Zhenfang, M., James, g., Gordon, S., and Gilbert, G. L. (2000) Molecular genotyping of human Ureaplasma species based on multiple-banded antigen (MBA) gene sequences. Internat J System Evolut Microbiol., 50, 1921-1929.

Kundsin, R. B., Driscoll, S. G., Monson, R. R., et al. (1984) Association of Ureaplasma urealyticum in the placenta with perinatal morbidity and mortality. N Engl J. Med., 310, 941-945.
Montagut, J. M., Lepretre, S., Degoy, J., et al. (1991) Ureaplasma in semen and IVF. Hum Reprod., 6, 727-729.
Quinn, P. A., Rubin, S., Nocilla, D. M., et al. (1983) Serological evidence of Ureaplasma urealyticum infection in neonatal respiratory disease. Yale J Biol Med., 56, 565-572.
Quinn, P. A., Butany, J., Taylor, J., et al. (1987) Chorioamnionitis: its association with pregnancy outcome and microbial infection. Am J Obstet Gynecol., 156, 379-387.
Quinn, P. A., Li, H. C., Th'ng, C., et al. (1993) Serological response to Ureaplasma urealyticum in the neonate. Clin Infect Dis., 17 Suppl 1, S136-143.
Riedel, H. H., Langenbucher, H. and Mettler, L. (1986) Significance of sperm bacteriology for the in vitro fertilization of human and mouse oocytes. J Reprod Med., 31, 605-608.
Shalika, S., Dugan, K., Smith, R. D., et al. (1996) The effect of positive semen bacterial and Ureaplasma cultures on in-vitro fertilization success. Hum Reprod., 11, 2789-2792.
Shepard, M. C., and Lunceford, C. D. (1978) Serological typing of Ureaplasma urealyticum isolates from urethritis patients by an agar growth inhibition method. J Clin Microbiol., 8, 566-574.
Taylor-Robinson, D. (1986) The male reservoir of Ureaplasma urealyticum. Pediatr Infect Dis., 5, S234-235.
Teng, L. J., Zheng, X., Glass, J. I., et al. (1994) Ureaplasma urealyticum biovar specificity and diversity are encoded in multiple-banded antigen gene. J Clin Microbiol., 32, 1464-1469.
Zheng, X., Teng, L-J., Watson, H. L., Glass, J. I., Blanchard, A. and Cassell, G. H. (1995) Small repeating units within the Ureaplasma urealyticum MB antigen gene encode serovar specificity and are assoicated with antigen size variation. Infect Immun., 63, 891-898.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

1. A method for detecting the presence of a reproductive function-modulating organism, comprising detecting an organism which is capable of adhering to sperm and which correlates with at least one of a presence and risk of a condition selected from the group consisting of male infertility, an adverse pregnancy outcome and an adverse assisted reproductive technology (ART) outcome, wherein the correlation is made if the organism is capable of remaining adherent to sperm after washing a sample comprising organism-positive sperm to permit one of removal and separation of non-sperm substances from the sample.

2. (canceled)

3. The method of claim 1, wherein the correlation is made if the washed sperm sample comprises at least 0.01% of the total number of organisms present in the sperm sample before washing.

4. The method of claim 1, wherein the washing is carried out using wash conditions that protect one or more viability properties of the sperm.

5. The method of claim 3, wherein the washing is carried out using wash conditions that permit continuation of adherence of Ureaplasma parvum serotype 6 to the sperm but that do not permit continuation of adherence of U. parvum serotype 1 to the sperm.

6. The method of claim 1, wherein the washing is carried out using a sperm swim-up technique.

7. The method of claim 1, wherein the washing is carried out using gradient centrifugation.

8. The method of claim 1, wherein the organism is selected from the class Mollicutes.

9. The method of claim 1, wherein the organism is selected from the order Mycoplasmatales.

10. The method of claim 1, wherein the organism is selected from the family Mycoplasmataceae.

11. The method of claim 1, wherein the organism is an ureaplasma.

12. The method of claim 1, wherein the organism is selected from the genus Ureaplasma.

13. The method of claim 12, wherein the organism is selected from the group consisting of a subtype of a strain and serotype selected from the group consisting of Ureaplasma parvum and Ureaplasma urealyticum.

14. The method of claim 12, wherein the organism is a subtype of Ureaplasma parvum serotype 6.

15. The method of claim 1, wherein the organism is selected from the genus Mycoplasma.

16. The method of claim 15, wherein the organism is selected from the group consisting of a subtype of a strain or serotype of Mycoplasma hominis.

17. The method of claim 1, wherein the adverse pregnancy outcome is selected from the group consisting of miscarriage, pre-term delivery, premature onset of labor, prolonged rupture of membranes, neonatal morbidity and mortality.

18. The method of claim 1, wherein the adverse ART outcome is selected from the group consisting of reduced embryonic development, reduced implantation rate, reduced fertilization rate, reduced clinical pregnancy rate, reduced viable pregnancy rate, reduced blastocyst culture rate and increased miscarriage rate.

19. (canceled)

20. (canceled)

21. (canceled)

22. A method for detecting an organism associated with at least one of male infertility and an increased risk of male infertility, comprising detecting an organism which is capable of adhering to sperm and which correlates with at least one of a presence and the risk of male infertility, wherein the correlation is made if the organism is capable of remaining adherent to sperm after washing a sample comprising organism-positive sperm to permit one of removal and separation of non-sperm substances from the sample.

23. A method for detecting an organism associated with at least one of an adverse pregnancy outcome and an increased risk of an adverse pregnancy outcome, comprising detecting an organism which is capable of adhering to sperm and which correlates with at least one of a presence and the risk of the adverse pregnancy outcome, wherein the correlation is made if the organism is capable of remaining adherent to sperm after washing a sample comprising organism-positive sperm to permit one of removal and separation of non-sperm substances from the sample.

24. A method for detecting an organism associated with at least one of an adverse assisted reproductive technology (ART) outcome and an increased risk of an adverse ART outcome, comprising detecting an organism which is capable of adhering to sperm and which correlates with at least one of a presence and the risk of the adverse ART outcome, wherein the correlation is made if the organism is capable of remaining adherent to sperm after washing a sample comprising organism-positive sperm to permit one of removal and separation of non-sperm substances from the sample.

25. A method for detecting at least one of a presence and diagnosing a risk of infertility in a male patient, comprising detecting in a sperm sample obtained from the patient an organism which is capable of adhering to sperm and which correlates with at least one of the presence and the risk of male infertility, wherein the correlation is made if the organism is capable of remaining adherent to sperm after washing a sample comprising organism-positive sperm to permit one of removal and separation of non-sperm substances from the sample.

26. A method for detecting at least one of a presence and diagnosing a risk of an adverse pregnancy outcome in a patient, comprising detecting an organism in a sperm sample obtained from a sperm donor of the patient, wherein the organism is capable of adhering to sperm and correlates with at least one of the presence and the risk of an adverse pregnancy outcome, wherein the correlation is made if the organism is capable of remaining adherent to sperm after washing a sample comprising organism-positive sperm to permit one of removal and separation of non-sperm substances from the sample.

27. A method for detecting at least one of a presence and diagnosing a risk of an adverse assisted reproductive technology (ART) outcome, comprising detecting the presence of an organism in a sperm sample used for the ART, wherein the organism is capable of adhering to sperm and correlates with at least one of the presence and the risk of an adverse ART outcome, wherein the correlation is made if the organism is capable of remaining adherent to sperm after washing a sample comprising organism-positive sperm to permit one of removal and separation of non-sperm substances from the sample.

28. The method of claim 27, wherein the ART is selected from the group consisting of artificial insemination (AI), in vitro fertilization (IVF) and intracytoplasmic sperm injection (ICSI).

29. A method for detecting at least one of a presence of and diagnosing a risk of a condition selected from the group consisting of male infertility, an adverse pregnancy outcome and an adverse assisted reproductive technology (ART) outcome in a patient, comprising detecting an organism in a biological sample obtained from the patient, wherein the organism is capable of adhering to sperm and correlates with at least one of the presence and the risk of the condition, wherein the correlation is made if the organism is capable of remaining adherent to sperm after washing a sample comprising organism-positive sperm to permit one of removal and separation of non-sperm substances from the sample.

30. The method of claim 29, wherein the biological sample comprises a biological fluid selected from the group consisting of whole blood, serum, plasma, saliva, urine, sweat, ascitic fluid, peritoneal fluid, synovial fluid, cerebrospinal fluid, amniotic fluid, seminiferous tubule fluid, semen, vaginal secretions, endocervical secretions, respiratory secretions, endometrial lining, fallopian tube washings, and follicular fluid.

31. The method of claim 29, wherein the biological sample comprises a tissue biopsy of at least one tissue selected from the group consisting of spermatozoan, endocervical cell, placental tissue, endometrial biopsy, chorioamnion, seminiferous tubule, ovarian tissue, oocyte, embryo, single cells biopsied from embryo, and fallopian tube tissue.

32. A method for diagnosing a higher risk of infertility in a male patient, comprising detecting in a biological sample obtained from the patient an organism which is capable of adhering to sperm and which correlates with a higher risk of male infertility, wherein the correlation is made if the organism is capable of remaining adherent to sperm after washing a sample comprising organism-positive sperm to permit one of removal and separation of non-sperm substances from the sample.

33. A method for diagnosis of a higher risk of an adverse pregnancy outcome in a patient, comprising detecting an ureaplasma in a biological sample obtained from a sperm donor of the patient, wherein the organism is capable of adhering to sperm and correlates with a higher risk of an adverse pregnancy outcome, wherein the correlation is made if the organism is capable of remaining adherent to sperm after washing a sample comprising organism-positive sperm to permit one of removal and separation of non-sperm substances from the sample.

34. A method for diagnosis of a higher risk of an adverse assisted reproductive technology (ART) outcome using sperm of a patient, comprising detecting an organism in a biological sample obtained from the patient, wherein the organism is capable of adhering to sperm and correlates with a higher risk of an adverse ART outcome, wherein the correlation is made if the organism is capable of remaining adherent to sperm after washing a sample comprising organism-positive sperm to permit one of removal and separation of non-sperm substances from the sample.

35. An isolated adhesin, wherein the adhesin is obtainable from an organism which is capable of adhering to sperm and which correlates with at least one of a presence and risk of a condition selected from the group consisting of male infertility, an adverse pregnancy outcome and an adverse assisted reproductive technology (ART) outcome, wherein the correlation is made if the organism is capable of remaining adherent to sperm after washing a sample comprising organism-positive sperm to permit one of removal and separation of non-sperm substances from the sample.

36. The adhesin of claim 35, which is capable of binding to a carbohydrate moiety on the surface of the sperm.

37. The adhesin of claim 35, which is capable of binding to a glycolipid.

38. The adhesin of claim 37, which is capable of binding to sulfogalactosylceramide.

39. The adhesin of claim 37, which is capable of binding to sulfatoxygalactosylceramide.

40. An isolated polynucleotide encoding the adhesin of claim 39.

41. A method for detecting an organism associated with at least one of male infertility and with an increased risk of male infertility, comprising interrogating a microbial sample for the presence of a gene encoding at least one of an adhesin and an expression product of the gene, wherein the adhesin is expressed by an organism which is capable of adhering to sperm and which correlates with at least one of a presence and risk of male infertility, wherein the correlation is made if the organism is capable of remaining adherent to sperm after washing a sample comprising organism-positive sperm to permit one of removal and separation of non-sperm substances from the sample.

42. A method for detecting an organism associated with at least one of male infertility and an increased risk of male infertility, comprising interrogating a microbial sample for the presence of at least one of a gene encoding an adhesin and an expression product of the gene, wherein the at least one of the adhesin and the expression product is expressible by an organism which is capable of adhering to sperm and which correlates with at least one of a presence and risk of male infertility, wherein the correlation is made if the organism is capable of remaining adherent to sperm after washing a sample comprising organism-positive sperm to permit one of removal and separation of non-sperm substances from the sample.

43. A method for detecting an ureaplasma associated with at least one of an adverse pregnancy outcome and an increased risk of an adverse pregnancy outcome, comprising interrogating a microbial sample for at least one of a presence of a gene encoding an adhesin and an expression product of the gene, wherein at least one of the adhesin and the expression product is expressible by an organism which is capable of adhering to sperm and which correlates with at least one of the presence and risk of the adverse pregnancy outcome, wherein the correlation is made if the organism is capable of remaining adherent to sperm after washing a sample comprising organism-positive sperm to permit one of removal and separation of non-sperm substances from the sample.

44. A method for detecting an ureaplasma associated with at least one of an adverse assisted reproductive technology (ART) outcome and an increased risk of an adverse ART outcome, comprising interrogating a microbial sample for a presence of at least one of a gene encoding an adhesin and an expression product of the gene, wherein at least one of the adhesin and the expression product is expressible by an organism which is capable of adhering to sperm and which correlates with at least one of a presence and risk of the adverse ART outcome, wherein the correlation is made if the organism is capable of remaining adherent to sperm after washing a sample comprising organism-positive sperm to permit one of removal and separation of non-sperm substances from the sample.

45. A method of screening for an agent which modulates the adherence of an organism to spermatozoa, wherein the organism is capable of adhering to sperm and correlates with at least one of a presence and risk of a condition selected from the group consisting of male infertility, an adverse pregnancy outcome and an adverse assisted reproductive technology (ART) outcome, wherein the correlation is made if the organism is capable of remaining adherent to sperm after washing a sample comprising organism-positive sperm to permit one of removal and separation of non-sperm substances from the sample the method comprising:
(a) contacting with a test agent a preparation comprising at least one of:
(i) a polypeptide comprising an amino acid sequence corresponding to at least a biologically active fragment of the adhesin of claim 35, and
(ii) a polynucleotide comprising at least a portion of a genetic sequence that regulates the expression of the adhesin of claim 35 and that is operably linked to a reporter gene; and
(b) detecting a change in at least one of a level and functional activity of at least one of the polypeptide and an expression product of the reporter gene, relative to at least one of a reference level and functional activity in the absence of the test agent.

46. (canceled)

47. (canceled)

48. An antigen-binding molecule that is immuno-interactive with an adhesin that is expressible by an organism which is capable of adhering to sperm and which correlates with at least one of a presence and risk of a condition selected from the group consisting of male infertility, an adverse pregnancy outcome and an adverse assisted reproductive technology (ART) outcome, wherein the correlation is made if the organism is capable of remaining adherent to sperm after washing a sample comprising organism-positive sperm to permit one of removal and separation of non-sperm substances from the sample.

49. A method of detecting an adhesin in a biological sample, wherein the adhesin is expressible by an organism which is capable of adhering to sperm and which correlates with at least one of a presence and risk of a condition selected from the group consisting of male infertility, an adverse pregnancy outcome and an adverse assisted reproductive technology (ART) outcome, wherein the correlation is made if the organism is capable of remaining adherent to sperm after washing a sample comprising organism-positive sperm to permit one of removal and separation of non-sperm substances from the sample, the method comprising:
contacting the sample with the antigen-binding molecule of claim 48; and
detecting the presence of a complex comprising the antigen-binding molecule and the adhesin in the contacted sample.

50. A method for prognostic assessment of at least one of male infertility, adverse pregnancy outcome and adverse assisted reproductive technology (ART) outcome in a patient, comprising detecting in a biological sample obtained from the patient a gene encoding at least one of an adhesin and an expression product of the gene, wherein the adhesin is expressible by an organism which is capable of adhering to sperm and which correlates with at least one of a presence and risk of a condition selected from the group consisting of male infertility, an adverse pregnancy outcome and an adverse assisted reproductive technology (ART) outcome, wherein the correlation is made if the organism is capable of remaining adherent to sperm after washing a sample comprising organism-positive sperm to permit one of removal and separation of non-sperm substances from the sample.

51. A method for affecting an adhesin selected from the group consisting of masking an adhesin and for otherwise interfering with the binding of the adhesin to sperm, wherein the adhesin is expressible by an organism which is capable of adhering to sperm and which correlates with at least one of a presence and risk of a condition selected from the group consisting of male infertility, an adverse pregnancy outcome and an adverse assisted reproductive technology (ART) outcome, wherein the correlation is made if the organism is capable of remaining adherent to sperm after washing a sample comprising organism-positive sperm to permit one of removal and separation of non-sperm substances from the sample, the method comprising contacting the adhesin with an antigen-binding molecule that is immuno-interactive with the adhesin.

52. A method for modulating adherence of an organism to sperm, wherein the organism is capable of adhering to sperm and correlates with at least one of a presence and risk of a condition selected from the group consisting of male infertility, an adverse pregnancy outcome and an adverse assisted reproductive technology (ART) outcome, wherein the correlation is made if the organism is capable of remaining adherent to sperm after washing a sample comprising organism-positive sperm to permit one of removal and separation of non-sperm substances from the sample, the method comprising contacting the organism with an agent that modulates the adherence of the organism to spermatoza, wherein the agent is identified by the method of claim 45.

53. The method of claim 52, wherein the agent decreases at least one of the level and functional activity of the adhesin.

54. A composition for improving male fertility, comprising an agent that reduces at least one of a level and functional activity of an adhesin that is expressible by an organism which is capable of adhering to sperm and which correlates with at least one of a presence and risk of male infertility, wherein the correlation is made if the organism is capable of remaining adherent to sperm after washing a sample comprising organism-positive sperm to permit one of removal and separation of non-sperm substances from the sample, and optionally a pharmaceutically acceptable carrier.

55. A composition for enhancing propensity for a favorable pregnancy outcome, comprising an agent that reduces at least one of a level and functional activity of an adhesin that is expressible by an organism which is capable of adhering to sperm and which correlates with at least one of a presence and risk of an adverse pregnancy outcome, wherein the correlation is made if the organism is capable of remaining adherent to sperm after washing a sample comprising organism-positive sperm to permit one of removal and separation of non-sperm substances from the sample and optionally a pharmaceutically acceptable carrier.

56. A composition for enhancing propensity for a favorable assisted reproductive technology (ART) outcome, comprising an agent that reduces at least one of a level and functional activity of an adhesin that is expressible by an organism which is capable of adhering to sperm and which correlates with at least one of a presence and risk of an adverse ART outcome, wherein the correlation is made if the organism is capable of remaining adherent to sperm after washing a sample comprising organism-positive sperm to permit one of removal and separation of non-sperm substances from the sample, and optionally a pharmaceutically acceptable carrier.

57. A method for at least one of a treatment and prophylaxis of male infertility, comprising administering to a patient in need of such treatment an effective amount of an agent that reduces at least one of a level and functional activity of an adhesin that is expressible by an organism which is capable of adhering to sperm and which correlates with at least one of a presence and risk of male infertility, wherein the correlation is made if the organism is capable of remaining adherent to sperm after washing a sample comprising organism-positive sperm to permit one of removal and separation of non-sperm substances from the sample, optionally in the presence of a pharmaceutically acceptable carrier.

58. The method of claim 57, wherein the agent is at least one of an antisense oligonucleotide and a ribozyme that interacts specifically with a polynucleotide encoding at least one of the adhesin and complement thereof.

59. The method of claim 57, wherein the agent is an antigen-binding molecule that is immuno-interactive with the adhesin.

60. A method for at least one of a treatment and prophylaxis of male infertility, comprising administering to the sperm of a patient in need of such treatment an effective amount of an agent that reduces at least one of a level and functional activity of an adhesin that is expressible by an organism which is capable of adhering to sperm and which correlates with at least one of a presence and risk of male infertility, wherein the correlation is made if the organism is capable of remaining adherent to sperm after washing a sample comprising organism-positive sperm to permit one of removal and separation of non-sperm substances from the sample, optionally in the presence of a pharmaceutically acceptable carrier.

61. The method of claim 60, wherein the agent is at least one of an antisense oligonucleotide and a ribozyme that interacts specifically with a polynucleotide encoding at least one of the adhesin or complement thereof.

62. The method of claim 60, wherein the agent is an antigen-binding molecule that is immuno-interactive with the adhesin.

63. A method for enhancing propensity for a favorable pregnancy outcome in a patient, comprising administering to a sperm donor of the patient an effective amount of an agent that reduces at least one of a level and functional activity of an adhesin that is expressible by an organism which is capable of adhering to sperm and which correlates with at least one of a presence and risk of an adverse pregnancy outcome, wherein the correlation is made if the organism is capable of remaining adherent to sperm after washing a sample comprising organism-positive sperm to permit one of removal and separation of non-sperm substances from the sample optionally in the presence of a pharmaceutically acceptable carrier.

64. The method of claim 63, wherein the agent is at least one of an antisense oligonucleotide and a ribozyme that interacts specifically with a polynucleotide encoding at least one of the adhesin and complement thereof.

65. The method of claim 63, wherein the agent is an antigen-binding molecule that is immuno-interactive with the adhesin.

66. A method for enhancing propensity for a favorable pregnancy outcome in a patient, comprising administering to the sperm of the patient's sperm donor an effective amount of an agent that reduces at least one of a level and functional activity of an adhesin that is expressible by an organism which is capable of adhering to sperm and which correlates with at least one of a presence and risk of an adverse pregnancy outcome, wherein the correlation is made if the organism is capable of remaining adherent to sperm after washing a sample comprising organism-positive sperm to permit one of removal and separation of non-sperm substances from the sample, optionally in the presence of a pharmaceutically acceptable carrier.

67. A method for enhancing propensity for a favorable assisted reproductive technology (ART) outcome in a patient, comprising administering to the sperm of the patient's sperm donor an effective amount of an agent that reduces at least one of a level and functional activity of an adhesin that is expressible by an organism which is capable of adhering to sperm and which correlates with at least one of a presence or risk of an adverse ART outcome, wherein the correlation is made if the organism is capable of remaining adherent to sperm after washing a sample comprising organism-positive sperm to permit one of removal and separation of non-sperm substances from the sample optionally in the presence of a pharmaceutically acceptable carrier.

68. The method of claim 66, wherein the agent is at least one of an antisense oligonucleotide and a ribozyme that interacts specifically with a polynucleotide encoding at least one of the adhesin and complement thereof.

69. The method of claim 66, wherein the agent is an antigen-binding molecule that is immuno-interactive with the adhesin.

70. A method for enhancing propensity for a favorable assisted reproductive technology (ART) outcome in a patient, comprising administering to the sperm of the patient's sperm donor an effective amount of an agent that reduces at least one of a level and functional activity of an adhesin that is expressible by an organism which is capable of adhering to sperm and which correlates with at least one of a presence and risk of an adverse ART outcome, wherein the correlation is made if the organism is capable of remaining adherent to sperm after washing a sample comprising organism-positive sperm to permit one of removal and separation of non-sperm substances from the sample.

71. The method of claim 70, wherein the agent is at least one of an antisense oligonucleotide and a ribozyme that interacts specifically with a polynucleotide encoding at least one of the adhesin and complement thereof.

72. The method of claim 70, wherein the agent is an antigen-binding molecule that is immuno-interactive with the adhesin.

73. An immunopotentiating composition for eliciting production of an element that specifically binds to an organism which is capable of adhering to sperm and which correlates with at least one of a presence and risk of a condition selected from the group consisting of male infertility, an adverse pregnancy outcome and an adverse assisted reproductive technology (ART) outcome, wherein the correlation is made if the organism is capable of remaining adherent to sperm after washing a sample comprising organism-positive sperm to permit one of removal and separation of non-sperm substances from the sample, the composition comprising at least one of a proteinaceous molecule that is an isolated adhesin of the organism and a vector including a polynucleotide encoding the proteinaceous molecule and operably linked to a regulatory polynucleotide, wherein the composition optionally further comprises a pharmaceutically acceptable carrier or adjuvant.

74. A method for at least one of a treatment and prophylaxis of male infertility, comprising administering to a patient in need of such treatment an immunogenically effective amount of the immunopotentiating composition of claim 73.

75. A method for at least one of a treatment and prophylaxis of an adverse pregnancy outcome in a patient, comprising administering to the patient's sperm donor an immunogenically effective amount of the immunopotentiating composition of claim 73.

76. A method for at least one of a treatment and prophylaxis of an adverse assisted reproductive technology (ART) outcome in a patient, comprising administering to the patient's sperm donor an immunogenically effective amount of the immunopotentiating composition of claim 73.

77. The method of claim 67, wherein the agent is at least one of an antisense oligonucleotide and a ribozyme that interacts specifically with a polynucleotide encoding at least one of the adhesin and complement thereof.

78. The method of claim 67, wherein the agent is an antigen-binding molecule that is immuno-interactive with the adhesin.


SEQUENCE LISTING (9 sequences)

SEQ ID NO: 1 - DNA, 17 bp, Artificial Sequence
  UMS -9 primer
attttttatattaggag

SEQ ID NO: 2 - DNA, 15 bp, Artificial Sequence
  UMA 163 primer
ttcaatgtcgtaaac

SEQ ID NO: 3 - DNA, 20 bp, Artificial Sequence
  UMS -81 primer
agaaattatgtaagattaat

SEQ ID NO: 4 - DNA, 28 bp, Artificial Sequence
  UMS-7 primer
atttcatatttagtttattaggsgatcg

SEQ ID NO: 5 - DNA, 26 bp, Artificial Sequence
  UMA 426 primer
ttcctggttgtgtttcaaaacctata

SEQ ID NO: 6 - DNA, 24 bp, Artificial Sequence
  UMA 429 primer
tgcctggttgtgtttcgaaactcc

SEQ ID NO: 7 - DNA, 24 bp, Artificial Sequence
  UMA 440 primer
cgttggttctggtgtatgagttgc

SEQ ID NO: 8 - DNA, 22 bp, Artificial Sequence
  UMA 442 primer
gttctggagttggtgtaggcgc

SEQ ID NO: 9 - DNA, 24 bp, Artificial Sequence
  UMA 461 primer
ttgaaccacttcctggttgtgtag